(19) United States
(12) Patent Application Publication
(10) Pub. No.: US 2008/0039879 A1
(43) Pub. Date: Feb. 14, 2008

(54) Devices and methods for atrial appendage exclusion

(76) Inventors: Albert K. Chin, Palo Alto, CA (US); John W. Davis, Sunnyvale, CA (US); Ketan Shroff, Pleasanton, CA (US); Amit Agarwal, San Francisco, CA (US); Shuji Uemura, San Francisco, CA (US); Alfredo R. Cantu, Pleasanton, CA (US); Geoffrey H. Willis, Mountain View, CA (US); Peter L. Callas, Castro Valley, CA (US); Charles Gresl, San Francisco, CA (US)

Correspondence Address: LAW OFFICE OF ALAN W. CANNON, 942 MESA OAK COURT, SUNNYVALE, CA, 94086, US

(21) Appl. No.: 11/502,249

(22) Filed: Aug. 9, 2006

(51) Int. Cl. A61B 17/08

(52) U.S. Cl. 606/157

(57) ABSTRACT

Devices, tools and methods for occluding fluid flow between two walls of tissue in a patient. Two walls of tissue are compressed together with sufficient compressive force to prevent fluid flow between the two walls, while ensuring that the compressive force is not so great as to cause tissue necrosis. The devices, tools and methods may be carried out using minimally invasive surgical techniques, such as in reduced-access surgical sites. Devices, tools and methods are provided for occluding an atrial appendix.

### FIELD OF THE INVENTION

The field of the present invention is apparatus and methods for performing minimally invasive surgery, more particularly to cardiac procedures performed with minimally invasive surgical techniques and apparatus.

### BACKGROUND OF THE INVENTION

In patients undergoing therapy for atrial fibrillation, for example atrial ablation therapy, it is desirable to exclude the left atrial appendage from the circulatory path, such as by sealing off the appendage from the remainder of the atrial cavity, or removing the appendage from the atrium. Blood clots tend to form in the atrial appendage of a patient experiencing atrial fibrillation. Once formed, these clots have the potential of detaching from the left atrial appendage and travel elsewhere in the body via the circulatory system, thereby increasing the risk of stroke, embolism and/or other circulatory complications.

Even when atrial ablation is performed in an attempt to cure atrial ablation, atrial appendage exclusion is still generally performed. In the event that the atrial ablation procedure is unsuccessful, the potential of stroke and other complications mentioned above is reduced in the patient with continuing atrial fibrillation that has had the left atrial appendage excluded.

One current technique for excluding the left atrial appendage is by suturing along the base of the atrial appendage where it joins the main atrial chamber, thereby closing off the appendage to the flow of blood. While effective, this technique generally requires an open chest procedure, i.e., open heart surgery, as suturing an appendage closed is very difficult to perform in a closed-chest environment and is generally not attempted.

Other techniques that have been used include: placing a line of staples across the base of the appendage, or filling the appendage with a space occupying device to fill up the cavity otherwise available for blood to flow into, in an effort to prevent blood flow into the atrial appendage cavity, and ultimately, to prevent blood clot formation there. Staplers have been used in closed-chest procedures for atrial appendage exclusion. Endoscopic gastrointestinal anastomotic (GIA) staplers are what are presently used to perform closed chest left atrial appendectomy. A GIA stapler is used to place one or more lines of staples across the base of the appendage. However, difficulties present with use of this technique, as there is a tendency for the staples to tear into the friable tissue of the appendage and cause bleeding, requiring the chest to be opened to repair the damage to the torn appendage. Further, since staplers that are presently used for these procedures are not designed for use on an atrial appendage, but rather for gastrointestinal use, the closure force on the staples, as the staples are placed in the appendage, may not be suitable for the tissue to which the force is applied. Further, the tissue thickness of the walls of the appendage may differ significantly from tissue thicknesses that the stapler is designed to close, resulting either in tissue damage to the appendage by the applied staple drawing the tissue walls too close together and thus crushing them, or incomplete closure, resulting in a failure to completely close off the appendage to the flow of blood. Still further, a line of staples placed may leave small pouches of atrial appendage at each end of the staple line. These residual pouches may be a source of thrombus (clot) formation.

Space occupying devices that are currently used also tend to leave areas of the appendage exposed to the blood path (circulation), with potential thrombus formation, and are particularly susceptible to this when delivered under closed chest conditions, such as via catheter, for example.

There is a continuing need for techniques and devices for excluding an atrial appendage (left and/or right atrial appendage) using minimally invasive procedures (e.g., closed chest procedures). Techniques that do not require a median sternotomy or substantial thoracotomy would decrease morbidity as well as hospitalization time.

### SUMMARY OF THE INVENTION

Devices, tools and methods for occluding fluid flow between two walls of tissue in a patient are provided. Two walls of tissue are compressed together with sufficient compressive force to prevent fluid flow between the two walls, while ensuring that the compressive force is not so great as to cause tissue necrosis.

Device and tools are provided for occluding fluid flow between two walls of tissue using minimally invasive surgical techniques, such as in reduced-access surgical sites.

Devices, tools and methods are provided for occluding an atrial appendix.

In at least one embodiment, a device for occluding fluid flow between two walls of tissue in a patient includes a base configured to apply compressive force against a first of the two walls; and at least one opposing member configured to apply compressive force against a second of the two walls upon installing the device, wherein, when installed, the base and the at least one opposing member compress the two walls therebetween, and a gap of predetermined dimension is established between the base and the at least one opposing member to sufficiently compress the two walls to occlude fluid flow therebetween, while preventing an establishment of a compression force that meets or exceeds a compression force to cause tissue necrosis.

In at least one embodiment, a device for occluding fluid flow between two walls of tissue in a patient includes a springform clip comprising at least three arms of substantially equal length, formed by bending an elongated member of spring metal, wherein the arms are elastically deformable to insert the tissue walls therebetween, and have sufficient length to traverse an entire width of the tissue walls defining a passageway through which fluid flow occurs, wherein, upon releasing the arms from a deformed, open configuration, the arms elastically spring back toward an undeformed configuration to compress the tissue walls with sufficient force to occlude fluid flow therebetween, while preventing an establishment of a compression force that meets or exceeds a compression force to cause tissue necrosis.

In at least one embodiment, a device for occluding fluid flow between two walls of tissue in a patient and a tool for installing the device are provided, wherein the device includes a base configured to apply compressive force against a first of the two walls; at least one opposing member configured to apply compressive force against a second of the two walls upon installing the device, wherein, when installed, the base and the at least one opposing member compress the two walls therebetween, and a gap of predetermined dimension is established between the base and the at least one opposing member to sufficiently compress the two walls to occlude fluid flow therebetween, while preventing an establishment of a compression force that meets or exceeds a compression force to cause tissue necrosis. A tool provided is configured to interface with at least one of the base and at least one opposing member, to drive the device in compression against the tissue walls.

An assembly for performing an occlusion of fluid flow between two walls of tissue in a patient is provided, including a device configured to maintain the two walls under sufficient compression to prevent fluid flow therebetween; and a tool configured to guide installation of the device into a final configuration where the device maintains the two walls under sufficient compression.

An assembly for performing an occlusion of fluid flow between two walls of tissue in a patient is provided, including a multi-lumen endoscopic tool including an endoscope positionable in a lumen of said multi-lumen tool; a steerable suction tool positionable in another lumen of the multi-lumen tool such that a distal contact surface extends distally of a distal end of the multi-lumen tool; and a snare device insertable through another lumen of the multi-lumen tool, such that a snare at a distal end of the snare tool extends distally of the distal end of the multi-lumen tool.

A tissue wall coating device for minimizing bleeding caused by an incision or puncture of the tissue wall is provided, including a main body in the shape of an elastomeric sack being closed at one end and having an opening at a base portion end; and a ligature extending around the base portion end and configured to constrict the opening at the base portion end.

A method of performing an occlusion of fluid flow between two walls of tissue in a patient is provided that includes compressing the two walls together under compression force sufficient to prevent fluid flow between the two walls, but not so great to cause tissue necrosis; and maintaining the compression force by installation of a device configured to maintain the walls between at least two contact surfaces separated from one another by a gap of predetermined dimension.

A method of performing an occlusion of fluid flow between two walls of tissue in a patient through a small opening in the patient is provided, including: inserting a multi-lumen endoscopic tool through the small opening in the patient, toward a location of the two walls of tissue to be occluded; identifying the location of the two walls of tissue to be occluded by viewing through an endoscope inserted in one of the lumens of the multi-lumen tool, and maneuvering a distal end of the multi-lumen tool to a reduced-access sited containing the two walls of tissue; snaring the tissue walls with a snare extending at the distal end of a snare tool inserted through another lumen of the multi-lumen device; tightening the snare from a location outside of the patient; fixing the snare in the tightened configuration to maintain compression of the two walls; and cutting the fixed snare away from the snare device.

A method of reducing bleeding during a surgical procedure on an atrial appendix is provided, including: placing an elastomeric sack over the atrial appendix to form a slightly compressive interface between the sack and the atrial appendix; constricting a base opening of the sack by drawing on a ligature passing through the base opening, and thereby compressing the walls of tissue at the location contacted by the base opening; and fixing the ligature relative to the base opening to maintain the constriction.

These and other advantages and features of the invention will become apparent to those persons skilled in the art upon reading the details of the devices, tools and methods as more fully described below.

### BRIEF DESCRIPTION OF THE DRAWINGS

FIGS. 1A-1B illustrate open and closed configurations of a device for occluding an atrial appendage.

FIG. 1C shows a tool interfacing with tool interfaces on the device of FIGS. 1A-1B in the open configuration.

FIG. 1D shows a tool interfacing with tool interfaces on the device of FIGS. 1A-1B in the closed configuration.

FIG. 1E is a schematic representation of a sectional view of a device taken perpendicularly to a longitudinal axis of the device.

FIG. 1F-1G illustrate that a thickness of the device is relatively greater in one direction than in another, to render the device relatively inflexible/nonmalleable in bending about one axis, and bendable about another.

FIGS. 2A-2B show another example of a device according to the present invention.

FIGS. 2C-2E illustrate partial views of a device as in FIGS. 2A-2B, and showing alternative hinge structures.

FIG. 2F illustrates a device of the type shown in FIGS. 2A-2B, with an alternative locking mechanism.

FIG. 2G illustrates a device having a curved side and a straight side, to better tailor the device to the site where the device is installed.

FIGS. 2H-2I show a device having ribs provided to run substantially along the length of the elongated portions to reinforce the portions to prevent bending or deformation along the longitudinal axis of the device.

FIG. 2J shows a variation in the locking mechanism of a device.

FIG. 2K shows another variation of a hinge for a device.

FIG. 2L shows still another variation of a hinge for a device.

FIG. 2M shows a variation of a device in which one or both of elongated members or portions may be dished to provide additional rigidity.

FIGS. 3A-3C illustrate another device that may be used to occlude fluid flow between two tissue walls.

FIG. 3D shows a device employing a series of clips of the type shown in FIGS. 3A-3C.

FIG. 3E illustrates another variation of device of the types shown in FIGS. 3A-3D.

FIG. 4A shows another example of a device useful for performing ligation by holding walls of tissue together sufficiently in apposition to prevent fluid flow therebetween.

FIG. 4B illustrates the device of FIG. 4A being used to ligate two opposing tissue walls.

FIG. 4C shows a variation of the device of FIG. 4A.

FIG. 4D shows another variation of the device of FIG. 4A.

FIG. 4E is a cutaway view showing a central body having been inserted into an opening between two walls of tissue, with a pair of plates connectable to the central body to compress the tissue walls between the central body, and the plates, respectively.

FIG. 4F illustrates another variation of a device in which a living hinge connects elongated plates thereof.

FIG. 5A is an illustration showing installation of another example of a device for closing two walls of tissue together.

FIG. 5B is a sectional illustration of the device shown in FIG. 5A having been installed.

FIGS. 5C-5G are variations of the device described with regard to FIGS. 5A-5B.

FIG. 6A illustrates another example of a device that may be used for ligation of opposite tissue walls to cut off fluid flow therebetween.

FIG. 6B illustrates a variation of the device shown in FIG. 6A.

FIG. 6C illustrates installation of the device shown in FIG. 6B.

FIGS. 6D-6F illustrate another embodiment of a device usable for ligation of opposite tissue walls to cut off fluid flow therebetween.

FIG. 6G illustrates another embodiment of a device usable for ligation of opposite tissue walls to cut off fluid flow therebetween.

FIG. 6H is a sectional view of an arm of the device shown in FIG. 6G.

FIG. 6I illustrates a variation of the device shown in FIG. 6A.

FIG. 7A is a sectional illustration of another device useful for ligation of a flow path past two walls of tissue.

FIG. 7B is a sectional illustration of the device of FIG. 7A having been installed in the performance of a ligation.

FIG. 7C illustrates another device for closing together tissue walls and an apparatus used to install such device.

FIG. 7D illustrates a distal end portion of the tube of the apparatus in FIG. 7C.

FIG. 8A shows another example of a device useful for holding the tissue walls together sufficiently in apposition to prevent fluid flow therebetween.

FIG. 8B illustrates a variation of the device shown in FIG. 8A.

FIG. 8C illustrates a variation of the device shown in FIG. 8A.

FIG. 9A illustrates a technique for atrial appendage ligation that may be practiced with any of the different devices described herein.

FIG. 9B illustrates a technique for atrial appendage ligation that may be practiced with any of the different devices described herein.

FIG. 10 illustrates a sectional view of a device having been inserted between walls at the base of an atrial appendage.

FIGS. 11A-11C illustrate another device according to the present invention, together with an installation tool, and illustrate different times/steps during the installation of such a device.

FIGS. 12A-12C illustrate another device according to the present invention, together with an installation tool, and illustrate different times/steps during the installation of such a device.

FIG. 13A represents a delivery tool for a device according to the present invention.

FIG. 13B illustrates a device that may be delivered by the tool shown in FIG. 13A.

FIGS. 13C and 13D illustrate variations of the device shown in FIG. 13B.

FIGS. 13E-13F illustrate various stages of installation of a device using the tool shown in FIG. 13A.

FIGS. 13G and 13H illustrate a variation of the device and tool shown in FIGS. 13A-13B.

FIGS. 13I and 13J shown installation of another device using another tool according to the present invention.

FIG. 14A illustrates a suction manipulator that may be used with other tools, devices and methods described herein.

FIG. 14B illustrates another embodiment of a device usable for ligation of opposite tissue walls to cut off fluid flow therebetween.

FIG. 14C illustrates a tool useful for installation of the device shown in FIG. 14B.

FIG. 15A illustrates another embodiment of a device usable for ligation of opposite tissue walls to cut off fluid flow therebetween.

FIG. 15B illustrates a tool useful for installation of the device shown in FIG. 15A.

FIG. 15C is a sectional view of the tool of FIG. 15B having been clamped over tissues to be ligated.

FIG. 15D shows the device of FIG. 15A after installation into the tissue walls shown.

FIGS. 15E-15H show variations for removing a sharpened distal tip of the device of FIG. 15A, after installation.

FIG. 15I shows a variation of the device shown in FIG. 15A.

FIG. 16A shows the device of FIG. 15I, together with a tool for its installation.

FIG. 16B shows a proximal end view of the driver and device shown in FIG. 16A.

FIG. 16C shows the jaws of the tool from FIG. 16A locked in a clamped configuration over tissue walls upon which a ligation is to be performed.

FIG. 16D shows the device of FIG. 151 having been installed.

FIG. 17A illustrates another embodiment of an installation tool and device usable to perform a ligation of opposite tissue walls to cut off fluid flow therebetween.

FIG. 17B illustrates a variation of the tool shown in FIG. 17A.

FIG. 17C illustrates the device shown in FIGS. 17A and 17B having been installed through tissue walls.

FIG. 18A illustrates another device useful for ligating tissue walls to cut off fluid flow therebetween.

FIG. 18B illustrates a variation of the device shown in FIG. 18A.

FIG. 18C illustrates a device of the type shown in FIG. 18A or FIG. 18B after installation.

FIG. 18D illustrates another device useful for ligating tissue walls to cut off fluid flow therebetween.

FIG. 18E illustrates a device of the type shown in FIG. 18D after installation.

FIGS. 19A-19F illustrate a multi-lumen endoscopic assembly and tools used in the performance of a closed chest, left atrial appendix ligation.

FIGS. 20A-20E illustrate a tissue wall coating device and methods for use thereof.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a device” includes a plurality of such devices and reference to “the atrium” includes reference to one or more atria and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “open-chest procedure” refers to a surgical procedure wherein access for performing the procedure is provided by a full sternotomy or thoracotomy, a sternotomy wherein the sternum is incised and the cut sternum is separated using a sternal retractor, or a thoracotomy wherein an incision is performed between a patient's ribs and the incision between the ribs is separated using a retractor to open the chest cavity for access thereto.

The term “closed-chest procedure” or “minimally invasive procedure” refers to a surgical procedure wherein access for performing the procedure is provided by one or more openings which are much smaller than the opening provided by an open-chest procedure, and wherein a traditional sternotomy is not performed. Closed-chest or minimally invasive procedures may include those where access is provided by any of a number of different approaches, including mini-sternotomy, thoracotomy or mini-thoracotomy, or less invasively through a port provided within the chest cavity of the patient, e.g., between the ribs or in a subxyphoid area, with or without the visual assistance of a thoracoscope.

The term “reduced-access surgical site” refers to a surgical site or operating space that has not been opened fully to the environment for access by a surgeon. Thus, for example, closed-chest procedures are carried out in reduced-access surgical sites. Other procedures, including procedures outside of the chest cavity, such as in the abdominal cavity or other locations of the body, may be carried out as reduced access procedures in reduced-access surgical sites. For example, the surgical site may be accessed through one or more ports, cannulae, or other small opening(s). What is often referred to as endoscopic surgery is surgery carried out in a reduced-access surgical site.

### Devices, Tools and Methods

Atrial appendage management, and particularly left atrial appendage (LAA) management, is a critical part of the surgical treatment of atrial fibrillation. When using a minimally invasive approach (e.g., where surgical access is provided by thoracoscopy, mini-thoracotomy or the like), there is a high risk of complications such as bleeding when using contemporary atrial appendage management, as noted above. Further, exposure and access to the base of the atrial appendage to be treated is limited by the reduced-access surgical site. The present invention provides devices and methods for ligating or occluding an atrial appendage, which ligation or occlusion may be performed while the heart continues to beat, and wherein such ligation or occlusion methods may be preformed using a minimally invasive approach. Such procedures may be performed solely from an opening in the right chest, or may be performed from a single opening in the left chest, if desired by the surgeon performing the procedure.

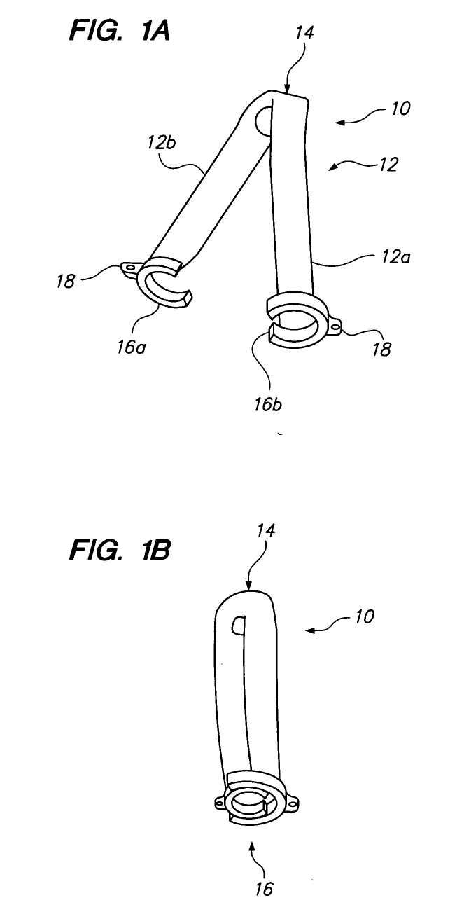

Referring now to FIG. 1A, an embodiment of a device 10 for occluding an atrial appendage is shown. Device 10 in this example comprises a clip that is configured to close over the base portion of the left atrial appendage to close off the atrial appendage to the flow of blood. Device 10 may come in a variety of dimensions to accommodate variations in the size of the atrial appendage base to be ligated. Device 10 may also be used to ligate the right atrial appendage, and the variations in dimension of device 10 may be advantageous to expand the range of tissues that may be ligated by device 10.

In this example, device 10 includes a malleable clip frame 12 having first and second portions or jaws 12a, 12b joined by a hinge 14 at one end of device 10. Locking mechanism 16 is provided at an end of device 10 opposite the end at which hinge 14 is formed. Locking mechanism may be formed from male and female features 16a, 16b configured to form a snap fit upon compressing them together, for example. Other mechanisms for automatically locking jaws 12a,12b together upon closing the jaws to relative positions as shown in FIG. 1B may be substituted, as would be readily apparent to one of ordinary skill in the art. Compressible material 18 lines the inside surfaces of jaws 12a,12b to provide a compliant clamping action against the outside surfaces of the base of an atrial appendage, when device 10 is closed and locked around such an appendage, thereby clamping the walls together and closing off the chamber within the atrial appendage from blood flow to or from the main chamber of the atrium from which the appendage extends. In the example shown, compliant material 18 is provided by elastomeric tubing slid over portions 12a,12b. Alternatively, layers of compressible material 18 may be formed or adhered to the inside surfaces of portions 12a,12b to add compliance to the clamping action. For example, a layer of compressible, open or closed-cell foam (e.g., made from an elastomeric material, such as silicone rubber, polyurethane, C-FLEX™ (silicone-based copolymer), or the like) may be adhered to the inner surface of each jaw 12a,12b. Alternatively, the compressible material 18 may be dovetailed into a slot in jaw 12a,12b to connect it thereto. FIG. 1B shows device 10 from FIG. 1A in a closed and locked configuration, the configuration that is maintained by device 10 around the base of an atrial appendage upon completion of a ligation procedure.

Tool interfaces 18 may be provided on portions 12a,12b to facilitate engagement of device 10 by a tool that is configured to actuate device 10 between an open and closed position, and which is further configured to lock device 10 by closing portions 12a,12b sufficiently to engage locking mechanism 16. In the example shown, tool interfaces 18 are loops extending from the ends of portions 12a,12b that are movable between open and closed orientations (i.e., end opposite hinge 14). While tool interfaces may be placed intermediately between the ends of portions 12a,12b, it is advantageous to place tool interfaces as close to locking mechanism 18 as possible to maximize the leverage of the loads that may be applied thereto by the tool used to manipulate device 10. The distal ends of a tool 20 are configured to be passed through tool interfaces 18 to provide tool 20 with positive control over the movements of device 10. For example, tool 20 has distal ends 22 that are angled (by a right angle bend or other angle) with respect to the remainder of instrument 20, and are dimensioned to be easily passed through loops 18 as shown in FIGS. 1C and 1D. In this example tool 20 includes a scissors joint 24 configured so that operation of handles 26 at the proximal end of instrument 20 effect the opening and closing of device 10 as the distal ends 22 are driven against tool interfaces 18 by operation of the proximal end portion of instrument 10.

Movement of instrument/tool 20 in a proximal or distal direction (i.e., in a direction toward the distal end of instrument 20 or toward the proximal end of instrument 20) also drives distal end portions 22 against tool interfaces 18 to at the same time move device 10 distally or proximally along with the distal or proximal movement of instrument 20, owing to the direct contact control of device 10 by instrument 20.

Device 10 may be configured to be malleable or bendable in one plane only by forming portions 12a,12b to have a relatively thin cross section in a dimension perpendicular to the axis of bending in the desired plane of bending while having a thicker cross section in directions aligned with the axis of bending. FIG. 1E is a schematic representation of a sectional view of device 10 taken perpendicularly to a longitudinal axis of device 10. In the example shown, bendability or malleability is desired over the length of device 10 about axis 30, for example. Accordingly, the thickness 32 of device 10 in a direction perpendicular to axis 30 is relatively thin, to allow device 10 to be bendable or malleable along its length about axis 30. On the other hand, the thickness 34 in the same direction as axis 30 is much greater to prevent bending about axis 38. This allows device 10 to be shaped (in malleable embodiments) or bent to conform to the base of an atrial appendage for even pressure and closure all along the base, thereby avoiding residual cavities in the appendage that may allow thrombus formation.

FIGS. 1F-1G are schematic illustrations of the entire device to further illustrate this principle, wherein it can be seen from the side view of FIG. 1G, that the thickness 32 over the length of the majority of device 10 is much less than the thickness at the distal end that forms locking mechanism 16, since locking mechanism needs to be relatively inflexible/non-malleable to function optimally for locking device 10. Comparatively, FIG. 1F shows that the thickness 34 of device 10 in a direction perpendicular to the direction of thickness 32 is relatively much thicker to make device 10 relatively inflexible/non-malleable in bending about the axis 38 in directions up and down on the paper on which FIG. 1F appears.

Alternatively, device 10 may be formed to be rigid and may be preformed with a curved shape that conforms to the contour present in the base of the atrial appendage to which it is to be applied. Upon placing portions 12a and 12b on opposite sides of the base of the atrial appendage and closing device 10 to lock portions 12a and 12b together as described above, device 10 clamps the base of the atrial appendage, thereby interrupting fluid communication between the atrial appendage and the main atrial cavity of the atrium from which the atrial appendage extends, without strangulating and necrosing the atrial appendage tissue at the site of the clamping.

Referring again to FIGS. 1C-1D, device 10 is shown engaged by the jaws of a surgical clamp 20 having been passed through tool interfaces 18, for use in inserting device 10 through a small thoracotomy incision, for example, and installing device 10 on the base of an atrial appendage. For insertion through the small opening in the patient, tool 20 may be manipulated to approximate the clip to a closed position, only not so far as to lock portions 12a,12b together. After passing through the small opening, tool 20 may be manipulated to separate portions 12a,12b further from each other, to or towards the open position illustrated in FIG. 1C. Once properly positioned approximate opposite faces of the base of the atrial appendage, tool 20 may then be used to clamp and lock portions 12a,12b together, in a manner as described above, thereby installing device 10 to clamp the base of the atrial appendage. Next, the jaws of tool 20 are removed from engagement with tool interfaces 18 and tool 20 is removed back through the small opening in the patient. Locking mechanism 16 may be made releasable, to allow curvature of device 10 to be varied/adjusted after initial placement and locking at the base of the appendage. Fitting 16a snaps into clasp 16b in a nested configuration during locking. By making the length of fitting 16a slightly longer than that of clasp 16b so that a distal portion of fitting 16b extends distally of the distal end of clasp 16b when in the locked configuration, a surgeon/user may then compress fitting 16a with an endoscopic grasper or other tool configured to compress, such as another tool functioning like pliers, to reduce the outside diameter of fitting 16a to release it from the friction fit with clasp 16b, thereby releasing it from clasp 16b.

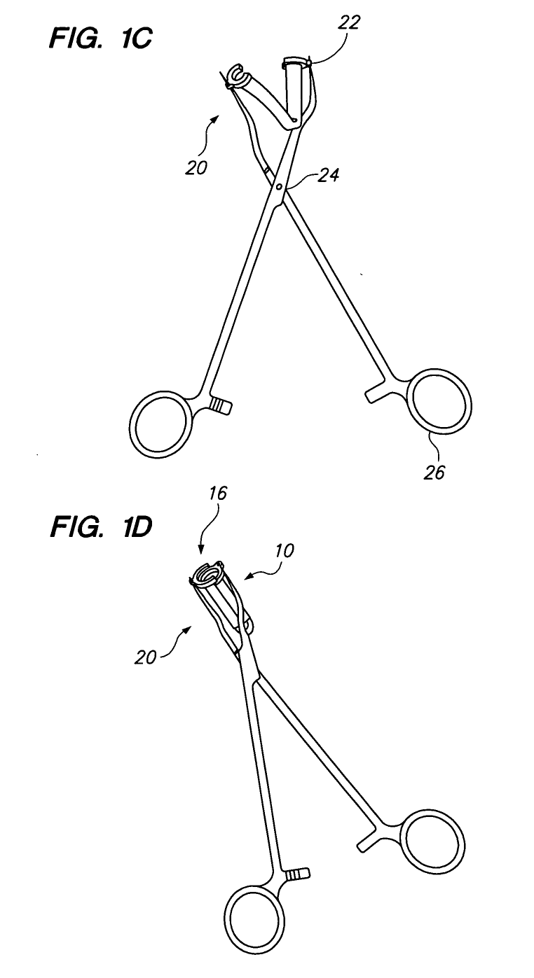

FIG. 2A shows another example of a device 10 for performing atrial appendage exclusion. Like the earlier described devices, device 10 is elongated to conform to the oval or oblong configuration of the base (or sometimes referred to as the “mouth”) of the atrial appendage to be clamped. Device 10 is substantially rigid and may be molded from biocompatible plastics or made of metal such as stainless steel or other known biocompatible, implantable metals. Hinge 14 in the embodiment shown in FIG. 2A includes a cylindrical or other curved proximal end portion of portion 12a that rotates within a mating portion 14b at the proximal end of 12b. Additionally, a tab 14t (see FIGS. 2A and 2B) may be provided at the proximal end portion of portion 12b that rides in a notch 14n (see also FIG. 2C) in the proximal end portion of portion 12a to maintain portions 12a and 12b in alignment during relative rotation of these components via hinge 14.

Hinge 14 may take other forms, as would be readily apparent to one of ordinary skill in the art. One example of an alternative hinge is shown in FIGS. 2D-2E where portion 12b is proved at a proximal end portion with a barrel or other cylindrically shaped portion 14b′ that is captured laterally by joint component 14a′ at the proximal end portion of portion 12a. Component 14a′ is also configured to conform to the curved surfaces of barrel 14b′ so that barrel 14b′ can rotate within component 14a′ By laterally constraining barrel 14b′, component 14a′ maintains portions 12a and 12b in alignment during relative rotation of these portions.

In order to prevent strangulation and subsequent necrosis of the atrial appendage tissue at the site of the clamping, device 10, when in a closed and locked configuration (such as is shown in FIG. 2A, for example) may maintain a gap 40 of predetermined width, to accommodate the combined thickness of the two tissue walls clamped therebetween, while providing adequate, but not excessive force to maintain the tissue walls in apposition to prevent fluid flow therebetween. For example, gap 40 may define a distance of about 0.02 inches to about 0.10 inches between the inner surfaces of portions 12a and 12b when in the locked configuration. The length of device 10 may typically range from about 1.5 inches to about 2.5 inches, more typically from about 1.75 inches to about 2.25″. However, since atrial appendages vary in size and wall thicknesses, the dimensions given are only typical examples, as devices 10 may need to be manufactured as a kit containing devices of varying lengths and with various gap thicknesses.

Locking mechanism 16 may be provided as a spring latch mechanism wherein one of portions 12a,12b (12a in the example of FIGS. 2A and 2F) is provided with a tang that extends further distally than latch 16b′ at the distal end portion of portion 12b (or portion 12a when tang is provided on portion 12b). Latch 16b′ may be ramped to act as a cam surface against which tang 16a′ rides as portions 12a and 12b are compressed together. Extension 16e is elastically deformable and is deformed by this action to allow tang 16a′ to pass the proximal edge of latch 16b′. Extension 16e then snaps back into its undeformed position such that the proximal edge of latch 16b′ again extends further proximally then the distal edge of tang 16a′, thereby preventing tang 16a′ from moving back past latch 16b′ and thus locking portions 12a and 12b together.

Alternatively to the configuration described above, device may not employ hinge 14 but instead may be configured with two locking mechanisms 16, one at each end of the device. Such a configuration may be preferred when a device 10 would be limited as to placement due to having one end already closed prior to positioning it in the location where clamping is desired. On the other hand, a device 10 which is already joined at one end by hinge 14 is easier to place since joint 14 positively maintains the alignment of portions 12a and 12b during placement, as already noted.

FIG. 2G shows a top view of the device 10 of FIG. 2A to more clearly illustrate that one side of portions 12a,12b may be formed straight, with the opposite respective sides 12c being formed with a curvature. This configuration allows flexibility in providing the best contoured fit to the shape of the atrial appendage to be clamped. When device 10 is applied near the base of an atrial appendage, a surgeon or other user has an option of installing device 10 so that curved side 12c or the straight side 12 is closer to the base of the appendage. This provides flexibility for more closely matching the contour of the base of the appendage against the contour of device 10. One or both of portions 12a,12b may also include one or more ribs 42 to reinforce that portion against bending or other deformation. In the example shown in FIGS. 2H-2I, ribs 42 are provided to run substantially along the length of portions 12a,12b to reinforce the portions to prevent bending or deformation along the longitudinal axis of device 10.

FIG. 2J shows a variation in locking mechanism 16, in which latch 16b′ is configured to be elastically deformable and is angled with respect to extension 16e in the undeformed configuration to overlap with the pathway of the distal end of tang 16a′. Thus, when portions 12a and 12b are closed against one another, tang 16a′ abuts against latch 16b′ and deforms it to allow tang 16a′ to move past latch 16b′. Latch 16b′ then snaps back into its undeformed position such that the proximal edge of latch 16b′ again extends further proximally then the distal edge of tang 16a′, thereby preventing tang 16a′ from moving back past latch 16b′ and thus locking portions 12a and 12b together.

FIG. 2K shows another variation of a hinge 16 that may be employed in device 10. One of portions 12a,12b (in this example, portion 12b) is provided with pegs, tabs or other extensions 44 that extend laterally from the proximal end portion thereof. The other portion (portion 12a in this example) is provided with brackets 46 extending from a proximal portion thereof. Brackets 46 have openings 46o therethrough designed to receive tabs 44 and allow rotation of tabs 44 with respect to brackets 46. However, brackets 46 laterally restrain portion 12b maintaining it in alignment with portion 12a.

FIG. 2L shows still another variation of hinge 16 that may be employed in device 10. One of portions 12a,12b (in this example, portion 12b) is provided with a ring or cylindrical portion 48 at a proximal end thereof, and the proximal end portion of the other portion 12a,12b (in this example 12a) is provided with an opening 50 though which ring or cylindrical portion 48 is assembled. The width of opening 50 is only slightly greater than the width of ring or cylindrical portion 48 to maintain portions 12a, 12b in alignment during rotation with respect to each other. Opening 50 freely passes over ring or cylindrical portion 48 during relative rotational movements between portions 12a and 12b.

FIG. 2M shows another variation of device 10 in which one or both of portions 12a,12b may be dished to provide additional rigidity, so that the side edges 12a′ act as longitudinal strengthening ribs. One or both portions 12a,12b may also be curved to better conform to the tissues to be compressed. One or more openings 12o may be provided in one or both of portions 12a,12b, such that when tissues are placed under compression between portions 12a and 12b, tissue protrudes somewhat through opening(s) 12o and this increased traction between device 10 and the tissue further ensures that the position of device 10 does not shift with regard to tissue 2.

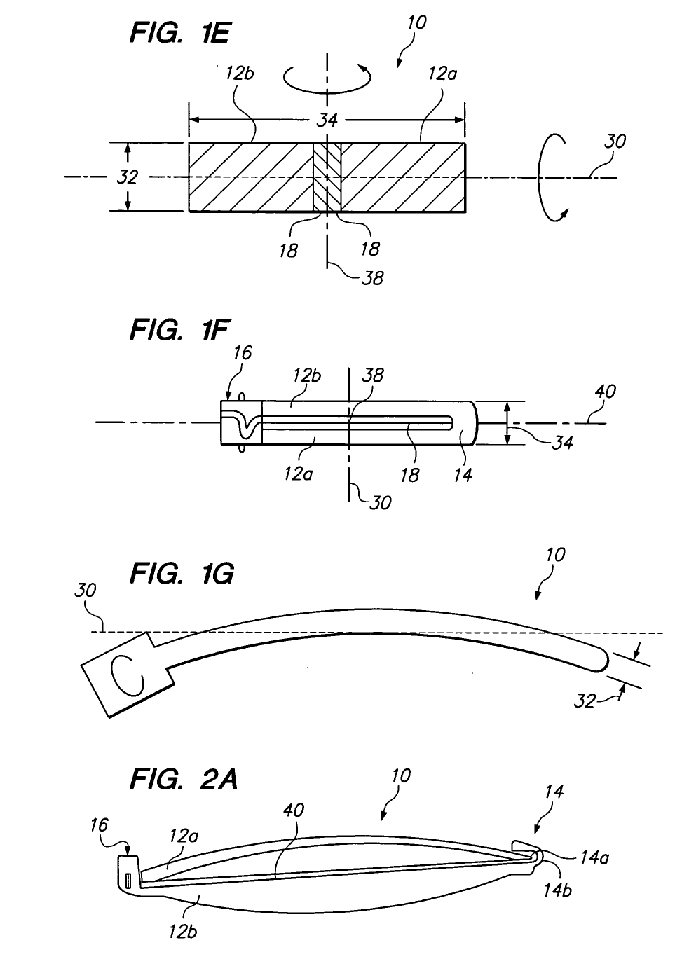

FIGS. 3A-3C illustrate device 10 used in an alternative approach to closing off fluid flow to an atrial appendage. Device 10 includes a malleable clip 52 with tissue piercing ends 54, such as barbs, pointed ends, sharpened ends of the like that are adapted to pierce through the tissue of the atrial appendage during placement. Clip 52 is typically made of metal such as stainless steel or other biocompatible, malleable metal, but may also be made from malleable polymers or composites that are biocompatible. Soft pads 56 are provided to be clamped to both sides of the atrial appendage to distribute point loads that would otherwise be applied to the tissue by simply clamping clip 52 to the tissue without such pads. Soft pads 56 may be made of cotton or other soft biocompatible material, and each pad may have a thickness of about 0.25 to 3.0 mm, typically about 0.5 to 1.0 mm for cotton and about 2.0 to 3.0 mm for softer materials. Soft pads 56 may include one or more openings 57 configured to allow tines 53 to pass therethrough without the need for ends 54 to pierce the material of soft pads 56. Alternatively, openings 57 may be omitted and ends 54 may be used to pierce through pads 56. A base 58 of the clip is provided with sufficient length to provide leverage to device 10 in the clamped configuration to prevent base 58 from pulling through the tissue being clamped. The length of base 58 typically varies from about 10 mm to about 30 mm. Kits of devices 10 having varying base lengths may be provided to account for variations in the lengths of tissue walls over which devices 10 are to be installed.

FIG. 3B illustrates a method of placing or installing devices 10 into the base of an atrial appendage 2 extending from an atrium 1. Device 10 is mounted on installation tool 60 so that one of soft pads 56 is temporarily fixed to one jaw 62 of tool 60. The other soft pad 56 is mounted over clip 52 and clip 52 is mounted to the opposite jaw 62 of tool 60. Clip 52 and upper pad 56 may be temporarily mechanically fixed to respective jaws 62, wherein the mechanical fixation is broken or released when tines 53 are bent over to lock device 10 into the compressed configuration shown on appendage 2 in FIG. 3B and jaws 62 are drawn apart/separated. Tool 60 includes a pivot joint 64 or other mechanism permitting jaws 62 to be driven together under force, similar to the action of pliers. Jaws 62 can be separated by the operator's manipulation of handles 66 to provide a distance between tips 54 and the opposite soft pad that is sufficient to receive the opposite walls of the base of atrial appendage (in an uncompressed state) therebetween.

Once mounted, device 10 is advanced by tool 60 so that the free pad 56 is positioned adjacent the base of atrial appendage 2 on one side of the base, and the other pad 56 with clip 52 are positioned adjacent the opposite side of the base. Jaws 62 are then compressed together via operation of handles 66, thereby driving the pointed ends 54 of clip 52 though the walls of the base of atrial appendage 2, through opening 57 of the opposite pad 56 and against the inner surface of the opposite jaw 62. The inner surface of opposite jaw 62 acts as an anvil against which tines 53 are driven by the compression action, thereby deforming tines 53 to fold over the opposite pad 56, clamping the walls of the base of atrial appendage 2 together in a fluid-tight seal between opposing pads 56. The inside surface of the top jaw 62 acts as an anvil as tine tips 54 are driven thereagainst. The inside surface may be angled from a most protruding ridge in the center of the jaw, in both directions toward the outside surfaces of the jaw (similar to the anvil of a stapler, except that the tine ends 54 initially contact the anvil near the center ridge and are then driven outwardly) to ensure that tines 53 are bent outwardly upon closing of the jaws 62. Additionally, or alternatively, tines 53 may already be slightly biased (not perpendicular to base 56) in outward directions to assure that the tines are bent outwardly upon being forced against the anvil surface of jaw 62. FIG. 3B illustrates two devices 10 having been already installed to close the walls of the base of atrial appendage 2 together.

FIG. 3C is an illustration of a sectional view of a portion of atrial appendage walls 2 having been closed together by the clamping force of device 10 upon installation. Tines 53 have been folded over to hold pads 56 and tissue walls 2 in compression against base 58 of clip 52. Tool 60 may be configured so that jaws 62 stop short of contacting one another, leaving a predefined gap therebetween to prevent over-compression of device 10 to prevent strangulation and necrosis of the tissue walls compressed therebetween. For example, tool 60 may be designed so that the inner surfaces of jaws bend tines 53 so that upon completion of deformation, a gap of predetermined width is maintained between the opposing faces of tines 53 and base 58. For example, the predetermined width may be from about 0.02 inches to about 0.16 inches, more typically about 0.04 inches to about 0.14 inches. However, tool 60 may be adjustable, or a kit of tools having varied predetermined stop distances may be provided to accommodate atrial appendage walls of different thicknesses, as well as soft tissue pads having different thicknesses. Further, kits of soft tissue pads having varying thicknesses may be provided.

Devices 10 may be installed adjacent one another, as close as desired, up to as close as an arrangement where adjacent clips abut one another, or even slightly overlap, if they are staggered enough so as not to interfere with one another as they are installed. Alternatively, devices 10 may be spaced apart slightly by a distance as determined sufficient by a surgeon performing the procedure. Further alternatively, device 10 may be formed to have a series of clips 52 with corresponding pads 56 adapted to be compressed therebetween, as illustrated in FIG. 3D. Device 10 may have a base 58 that joins all clips 52 together or multiple clips 52 may have separate bases and be separately inserted through pad 56 to hold them all together when placing them adjacent a wall of atrial appendage 2 to be installed. The overall length of multiple clip device 10 may vary, and may be as long as to nearly approximate the entire length of tissue wall to be closed off. Once installed to compress the tissue walls 2 together, the ends 54 of tines 53 from adjacent clips 52 may abut one another or even overlap one another slightly, along the length of the installations, with the devices being staggered in the width direction.

FIG. 3E illustrates another variation of device 10 wherein the clamping function provided operates similarly to device 10 described with regard to FIGS. 3A-3D, but where installation of clips 52 through tissue wall 2 varies somewhat. Tines 53 are angled or partially bent over at an acute angle with respect to the base 58 of clips 52 and pad 56, as shown in FIG. 3E. To install device 10, the clip portion, with one pad 56 mounted thereon (bottom portion shown in FIG. 3E is placed against a wall of the base of an atrial appendage 2 and slid laterally (i.e., in the direction shown by the arrow in FIG. 3E). During sliding, piercing tips 54 of tines 53 pierce the wall of the tissue applied thereto. This anchors the tips 54 with respect to the lateral movement. As the lateral movement is continued, this applies force to the tips 54, thereby straightening tines 53 with respect to base 58 and pad 56 (i.e., toward a perpendicular orientation of the tines with respect to the base). Lateral movement is continued until tines 53 are substantially perpendicular to pad 56 and base 58. Next, force is applied against device 10 in a direction substantially perpendicular to the walls of atrial appendage 2, on both base 28 and opposing pad 56, causing tines 53/tips 54 to completely pierce both walls of tissue 2, as well as the opposing pad 56 (in embodiments where openings 57 are not provided). Tines 53 are then deformed in a manner similar to any of those that described above with regard to FIGS. 3B and 3D.

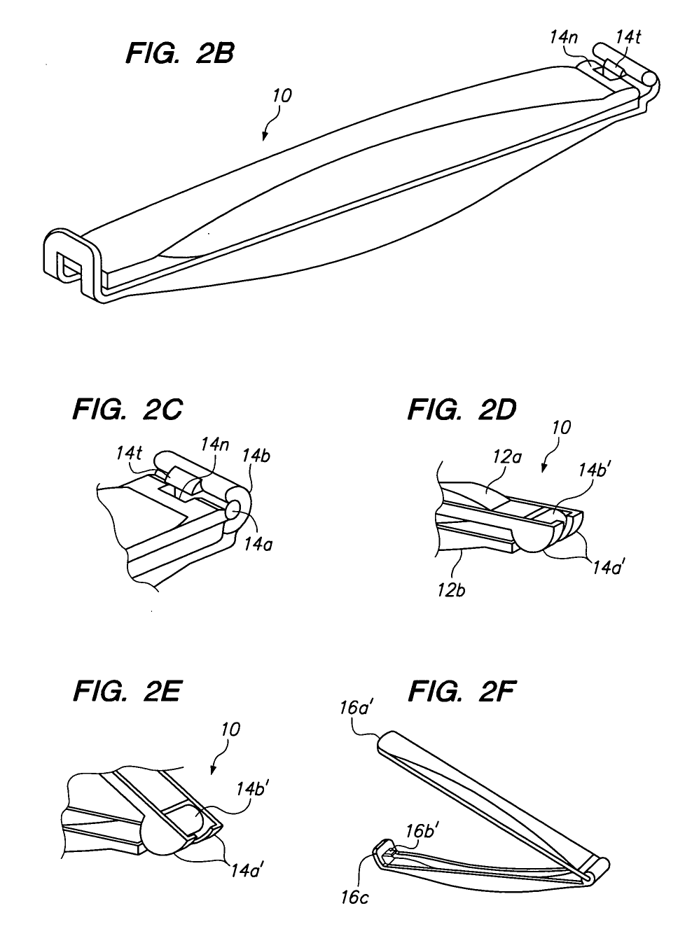

FIG. 4A shows another example of a device 10 useful for ligating an atrial appendage by holding the walls of the base of the atrial appendage sufficiently in apposition to prevent fluid flow therebetween. Device 10 includes two cooperating bodies or plates 70,72 designed to lock together to sandwich the walls of an atrial appendage 2 therebetween under compression to prevent blood flow into and out of the atrial appendage 2. Plates 70,72 may be rigid, or alternatively one or both may be malleable so as to be shaped to conform to the anatomy of the atrial appendage base. Further alternatively one or both may be flexible so that, when locked together, in a manner described hereafter, the one or more flexible plates conform to the surfaces of the atrial appendage walls for an optimum fit, but not so flexible as to be incapable of compressing the walls of tissue when compressed together by the locking action. Still further, device 10 may be made entirely or partially of one or more bioabsorbable materials (e.g., polylactic acid, polyglycolic acid, bioabsorbable glass, etc.) to remove any potential of long term negative effects of maintaining an implant at the site.

Connecting members 74 include barbed tips 76 designed to interface with and lock against the opposite side of plate 72 after passing through openings 78. Openings 78 have a circumference or other perimeter slightly less than the circumference or other perimeter of the greatest cross sectional area of barbs 76. In use, plates 70 and 72 are positioned adjacent opposite walls of an atrial appendage 2 to be ligated, at the base of the appendage in a location determined by the surgeon as the target area to perform the ligation. Tips 76 are aligned with openings 78 and plates 70 and 72 are then compressed together, causing tips 76 to pierce the tissue walls 2 and driving tips 76 through openings 78. The portion of tips 76 having the greatest cross-sectional areas, respectively may elastically deform under the driving force to enable them to pass through the slightly smaller openings 78. Once through, tips 76 elastically resume their unstressed configurations so that the faces 80 of the enlarged portions interface with and abut against the opposite face 72f of plate 72, as shown in FIG. 4B. The length 741 of connecting members 74 and thickness 72w of plate 72 together determine a distance d by which plates 72 and 70 are maintained after completion of the connection, and thus determine the amount of compression that tissue walls 2,2 are kept under. The length 741 and/or width 721 are modifiable to change the amount of desired compression, or to maintain the same desired amount of compression over procedures done on atrial walls having various thicknesses. For example, the length may be about 2.5±0.5 inches and the thickness may be about 0.09±0.06 inches. As noted, these dimensions may vary depending upon the particular application, and also upon the materials used. A kit of plates 72 having varying thicknesses and/or plates 70 having connecting members of varying lengths 741 may be provided to more flexibly and readily meet the demands of a particular procedure being conducted.

While any appropriate surgical tools may be used to provide the compressive forces against plates 70 and 72 during installation, e.g., surgical clamp tools may be used to individually fasten connectors 74 by pressing tips 76 though holes 78 one at a time, respectively, a tool 82, such as one with parallel motion jaws that function like pliers, with each jaw having a pocket to hold the respective plates, may be provided to maintain plates 70 and 72 in alignment, as well as to fasten all connectors simultaneously to join plates 70 and 72 together.

FIG. 4C illustrates a variation of the device 10 described above with regard to FIGS. 4A and 4B. In this arrangement, connectors are provided only at the ends of plate 70 and are configured with lips 74c or other features at the ends thereof to form a snap fit connection with plate 72. The length of plates 70 and 72 is sufficient to span the base of the atrial appendage, so that plates 72 and 70 in this instance are fastened “around” the base of the appendage, rather than through it. For example, the required length for spanning an atrial appendage is typically in the range of about two to three inches. In one example, the length was 2.25 inches. As in the previous discussion, the distance maintained between the opposing faces of plates 70,72 is determined by the length 741 of connectors 74 and thickness 72w of plate 72.

FIG. 4D shows a variation of the device of FIG. 4C wherein a living hinge 84 replaces one of the connectors 74 at one end of the plates 70,72. Device 10 is installed similarly to that of the device described with regard to FIG. 4C, but because one set of ends is already connected by living hinge 84, plates 70,72 are easier to maintain in alignment during installation, and only connector 74 needs to be connected via connecting mechanism 74c,74m and this is accomplished as a snap fit by simply pressing plate 70 into the position shown in phantom in FIG. 4D. Areas of lesser thickness or weakened areas, such as notches 84n or the like may be provided to facilitate the function of living hinge 84.

FIG. 4E is an illustration of a cutaway view showing installation of device 10 at the site of the base of an atrial appendage. Device 10 in this example, includes a pair of plates 70 having connectors 74 with barbed tips 76 extending therefrom. Plates and connectors may be made to have any of the characteristics described above with regard to plate 70 in FIG. 4A. Device 10 includes a third component in this example, an insertable body 86 having openings 78 that cooperate with each of barbed tips 76 to connect plates 70 to body 86.

Like plates 70, body 86 may be preformed and rigid, malleable or flexible to better conform to the opening of the atrial appendage to the atrium to prevent the shape of the atrium from being altered, and has a length equal to or slightly greater than an length of the opening of the atrial appendage to ensure sealing with the inner wall at the ends of the insert. An incision through the side of the atrial appendage may be made to insert body 86 at the opening to the atrial appendage (corresponding to the base of the walls of the appendage 2). FIG. 4E is a cutaway view showing body 86 having been inserted into the opening 3 of the appendage 2, with the atrium having been cut away in this view. Body 86 may be hollow and provided with openings 78 on two sides thereof to line up and connect with connectors 74 from each of plates 70,70 respectively. This is the arrangement shown in FIG. 4E. Alternatively, body 86 may be formed solid, wherein openings 78 pass entirely therethrough from one side to the other. In this case, connectors 74 of plates 70,70 may be formed in an alternating pattern, e.g., such that every other opening is engaged by a connector 74 from the same plate 70 and the other plate 70 engages the remaining openings 78.

FIG. 4F illustrates another variation of device 10 in which a living hinge 84 connects plates 70 and 72. Additionally, connectors 74 are each provided with a series of ratchet features such as barbs 75 dimensioned to interlock plates 70 and 72 after passing through openings 78. specifically, the smaller dimensioned leading portion of each ratchet feature has a smaller perimeter than an inside diameter or perimeter of corresponding opening 78 so as to easily pass therethrough. The trailing portion of each ratchet feature has a larger perimeter than the inside diameter or perimeter of corresponding opening 78. However, the trailing portion is deformable and deforms to pass through opening 78 when driven in the direction of the arrow in FIG. 4F. Once through the opening 78, the trailing portion expands to its undeformed dimension, preventing the ratcheting feature 75 from passing back though the opening in an opposite direction, thereby locking plates 70 and 72 together. The provision of multiple ratchet features 75 on each connector permits adjustment of the gap between plates 70 and 72, consequently adjusting the amount of compression placed on the tissue walls therebetween. Further, each connector 74 is independently adjustable, so that a variable gap may be created between the plates 70,72 to accommodate variations in tissue wall thicknesses along the lengths of plates 70,72.

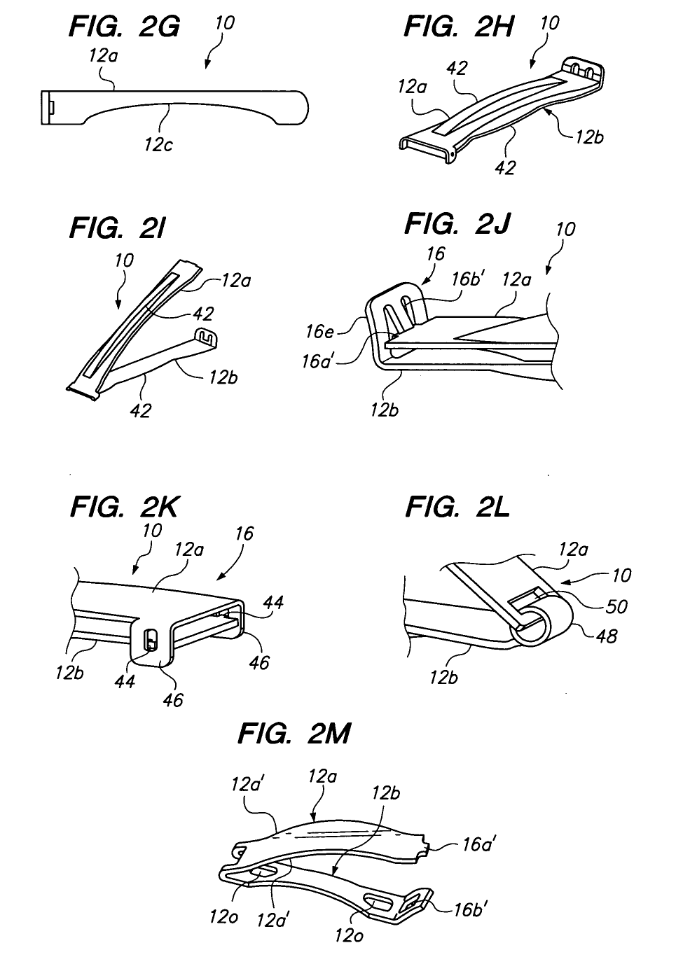

FIG. 5A is an illustration showing installation of another example of a device 10 for closing two walls of tissue together, particularly the walls at the base of an atrial appendage 2. Device 10 includes at least one malleable tine 53 (typically two, although more may also be provided, and the embodiment of FIG. 5F has only one) with tissue piercing end 54, such as barbs, pointed ends, sharpened ends of the like that are adapted to pierce through the tissue of the atrial appendage during placement. Device 10 is typically made of metal such as stainless steel or other biocompatible, malleable metal (e.g., thin wire or sheet metal), but may also be made from malleable polymers or composites that are biocompatible. Base 58 of device 10 is provided with sufficient length to provide leverage to device 10 in the clamped configuration to prevent base 58 from pulling through the tissue being clamped.

Installation of device 10 may be performed by mounting device 10 on a jaw 62 of installation tool 60 as shown in FIG. 5A. Mounting may be performed using clips or any other mechanical means that are broken or releasable after device 10 has been installed in a manner as described hereafter. Tool 60 includes a pivot joint 64 or other mechanism permitting jaws 62 to be driven together under force, similar to the action of pliers. Jaws 62 can be separated by the operator's manipulation of handles 66 to provide a distance between tips 54 and the inner surface of opposite jaw 62 that is sufficient to receive the opposite walls of the base of atrial appendage 2 (in an uncompressed state) therebetween. A typical configuration may provide a minimum clearance between the tissue wall received and the tips/jaw of the apparatus, of at least a few thousandths of an inch clearance up to several millimeters, typically about a millimeter of clearance. Once mounted, device 10 is advanced by tool 60 to a position where device 10 is located adjacent the base of atrial appendage 2 on one side of the base, and the other jaw 62 on which device 10 is not mounted, is positioned adjacent the opposite side of the base. Jaws 62 are then compressed together via operation of handles 66, thereby driving the pointed ends 54 and tines 53 through the walls of the base of atrial appendage 2, and against the inner surface of the opposite jaw 62. The inner surface of opposite jaw 62 acts as an anvil against which tines 53 are driven by the compression action, thereby deforming tines 53 to fold over the opposite wall of atrial appendage 2, thereby clamping the walls of the base of atrial appendage 2 together in a fluid-tight seal as illustrated in FIG. 5B. Similar to the embodiment of FIG. 3B assurance that the tines 53 will bend outwardly may be provided by the shaped of the anvil of the jaw of tool 60 (inner surface of bottom jaw of device 60 shown in FIG. 5A) and or the initial orientation of extension of tines 53 from base 58.

FIG. 5B is an illustration of a sectional view of atrial appendage walls 2 having been closed together by the clamping force of device 10 upon installation. Tines 53 have been folded over to hold tissue walls 2 in compression against base 58 of device 10. Tool 60 may be configured so that jaws 62 stop short of contacting one another, leaving a predefined gap therebetween to prevent over-compression of device 10 to prevent strangulation and necrosis of the tissue walls compressed therebetween. For example, tool 60 may include stop 88 that extends between handles 66 to prevent complete closure of tool 60. The distance by which stop 88 extends between handles 66 may be adjustable, such as by threadably engaging stop through one of handles 66 as shown. As such, the final gap between the inner surfaces of jaws 62 when lower handle 66 abuts stop 88 may be adjusted by turning stop either clockwise or counterclockwise to increase or decrease the final gap as desired. In this way a predetermined distance may be defined between the bent over tines 53 and base 58 resulting from deformation by tool 60, so that an appropriate amount of compression can be applied to the tissue walls 2. That is, the inner surfaces of jaws 62, bend tines 53 so that upon completion of deformation, a gap of predetermined width is maintained between the opposing faces of tines 53 and base 58. For example, the predetermined width may be from about 0.02 inches to about 0.16 inches, more typically about 0.04 inches to about 0.14 inches. However, this may be adjusted by adjusting stop 88, as noted, or a kit of tools having varied predetermined stop distances may be provided to accommodate atrial appendage walls of different thicknesses.

Devices 10 may be installed adjacent one another, as close as desired, up to as close as an arrangement where adjacent tine ends 54 abut one another, or even slightly overlap. Alternatively, devices 10 may be spaced apart slightly by a distance as determined sufficient by a surgeon performing the procedure.

FIGS. 5C-5G are illustrations of various device designs that operate in the manner described above with regard to FIGS. 5A-5B. In FIG. 5C, the length of base 58 is greater than the distance between tines 53 to provide additional leverage against the wall of tissue that base 58 abuts under compression. FIG. 5D shows a device 10 having a base 58 equal in length to the separation distance between tines 53. In FIG. 5E, tines 53 are bent or angled, so that base 58 is angled from the distal axes of tines 53. Device 10 of FIG. 5F has a spiral or circular base and a single tine 53. The variant of FIG. 5G is similar to that shown in FIG. 5E, but tines 53 are curved rather than angled.

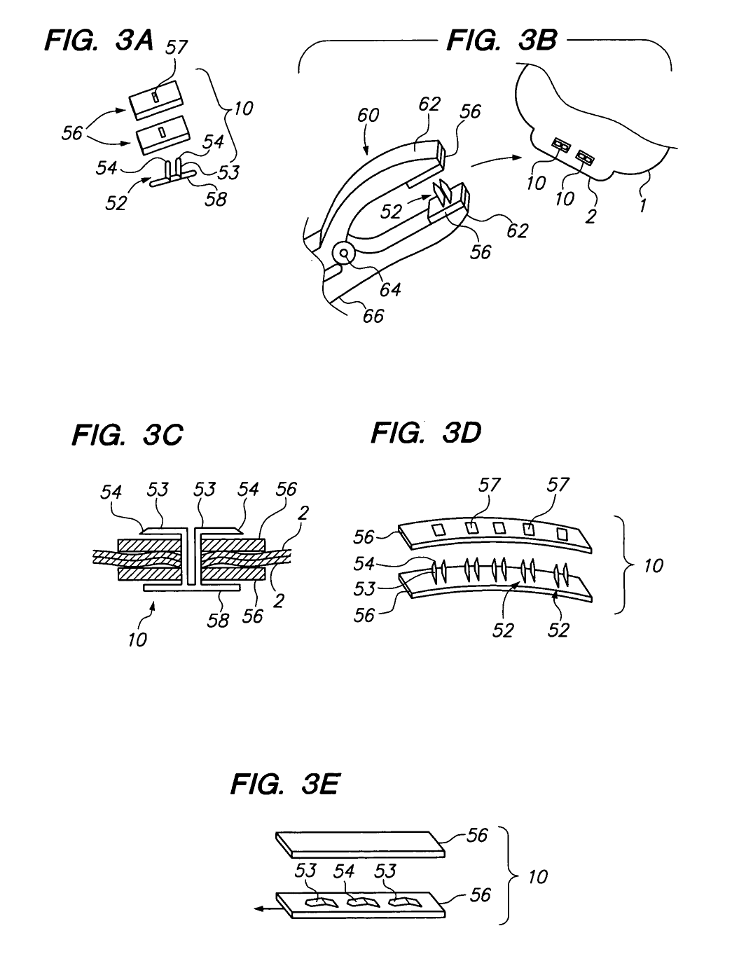

FIG. 6A illustrates another example of a device 10 that may be used for ligation of opposite tissue walls to cut off fluid flow therepast. In this example, device 10 is a springform device having three arms formed by bending of spring steel (e.g., surgical stainless steel) or other material or wire capable of undergoing elastic deformation to an extent sufficient to form a space between arms capable of receiving the opposite tissue walls therebetween, and which, when released, are capable of applying a spring force sufficient to compress the tissue walls together with sufficient force to create a fluid-tight seal therebetween. Additionally, arms 90 may be provided with traction features 92 such as nubs, barbs, knurling, or the like to grab the tissue walls, once placed between the opened arms 90, and during release and closing of arms 90 to ensure that the tissue 2 does not slip out from between arms 90 as they close and compress the tissue walls 2 together. To install the device, arms 90 are separated to provide clearance therebetween as shown in FIG. 6A, and device 10 is then slid over the tissue walls to be compressed by device 10. After properly positioning the device in the target location where the walls are desired to be brought into contact with one another, arms 90 are released and the spring force of arms 90 compresses the arms 90 against the outer walls of the tissue 2. Any instrument configured to engage arms 90 and pull them apart may be used for installation, e.g., graspers, forceps, etc. Optionally, engagement members (not shown, but such as loops 18 shown in FIG. 1A, for example) may be provided on arms 90 to facilitate drawing the arms 90 apart.

FIG. 6B illustrates a device 10 similar to that described above with regard to FIG. 6A, although formed to have four arms 90. Device 10 may be made of any of the same materials described with regard to the device of FIG. 6A and to have the same characteristics. By pulling on the ends 94 of device 10 in directions indicated by the arrows in FIG. 6C, the inner arms 90 of device 10 can be separated to form a space sufficient so that device 10 can be positioned over the walls of tissue 2 to be ligated, as shown in FIG. 6C. Installation of the device 10 shown in FIG. 6B may be performed in the same manner as described above with regard to installation of device 10 in FIG. 6A. Then, upon release of ends 94 arms 90 spring back toward the conformation shown in FIG. 6B, thereby compressing the walls 2 of tissue together to close off fluid flow therebetween. Although not shown, arms 90 of device 10 in FIGS. 6B-6C may also be provided with traction features 92 to prevent backsliding of the tissue wall with respect to arms 90 as they are compressing the tissue walls. Alternative devices according to the previously described concepts may have more than four arms. A large number of arms may be included in a spring form device and adapted to apply compressive forces to tissue walls placed therebetween at multiple locations along the device. For example, FIG. 61 shows a device 10 having ten arms 90, adapted to apply compressive forces at four different locations along device 10 between different pairs of arms 90.

FIGS. 6D-6F illustrate another variation of a springform device 10 in which two clamping arms 98 are interconnected by torsion arms 96, wherein when arms 98 are opened, or separated from one another, arms 96 are twisted or torqued about the rotational axes indicated by the arrows in FIG. 6D. Torsion arm 96 are thus elastically deformed under torsion, and store potential energy under such torsion that is converted to kinetic energy when the opening forces on the compression or clamping arms 98 are released, thereby returning device toward the configuration shown in FIG. 6D. Device 10 may be configured so that clamping arms, in their undeformed positions, are separated by a gap of predetermined width. For example, the predetermined width may be from about 0.02 inches to about 0.16 inches, more typically about 0.04 inches to about 0.14 inches, and may be tailored to provide compression to the tissue wall captured therebetween, with sufficient force to form a fluid tight seal between the walls, but not so great as to cause necrosis.

To install device 10, arms 98 are opened to provide a gap 68g (FIG. 6E) therebetween sufficient to allow device 10 to be positioned such that opposite arms 98 are placed adjacent opposite walls of an atrial appendage 2. Then, the forces that were applied to open arms 98 are released and torsion arms 96 snap back (twist back) to their unbiased configurations, causing arms 98 to clamp the walls 2 together, thereby closing off the atrial appendage to fluid flow, as illustrated in FIG. 6F.

FIG. 6G illustrates a variation of device 10 shown in FIGS. 6D-6F. In this variation, device 10 is provided with a torsion bar 100 adapted to store potential energy as it is rotationally deformed in the directions indicated by the arrows in FIG. 6B. Side arms 102 and clamping arms 104 are configured to be rigid in the directions of rotation and to resist bending when clamping forces are applied to the tissue walls. For example, arms 102 and 104 may be much wider than they are thick, as illustrated in the sectional view of arm 104 in FIG. 6H, wherein width 104w is much greater than thickness 104t.

Forces are applied to arms 104 to move them to the open configuration, much in the same manner as described with regard to the previous variation. However, arms 104,102 do not bend or twist, but translate the opening forces to torsion bar 100 which undergoes the torsional deformation and storage of potential energy. Device 10 is installed in the same manner as described previously with regard to the device in FIGS. 6D-6E.

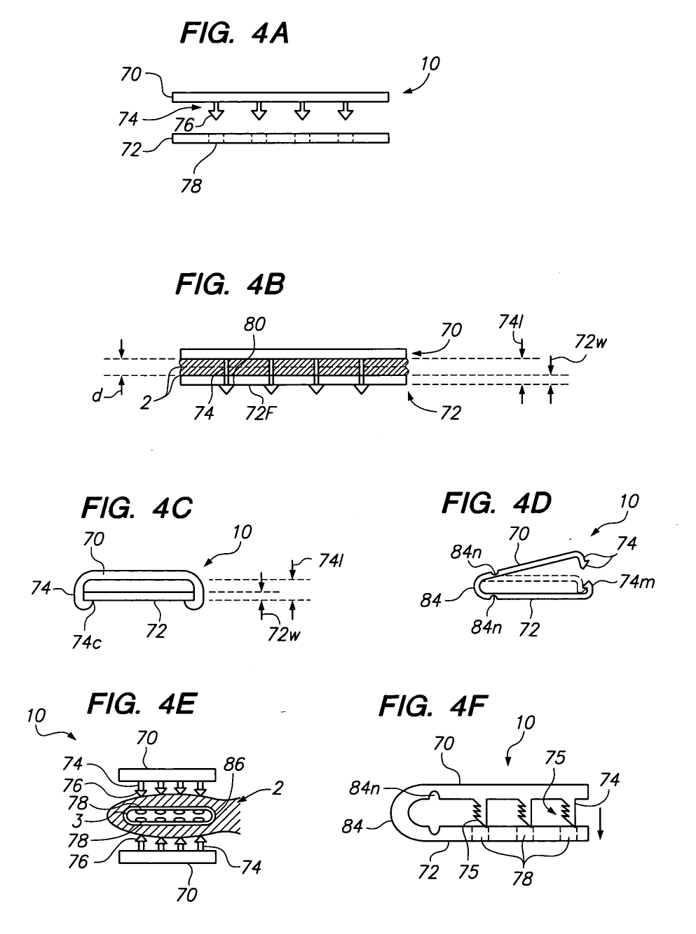

FIG. 7A is a sectional illustration of another device useful for ligation of a flow path past two walls of tissue, such as at the base of an atrial appendage, for example. Device 10 includes a spike 106 having a tissue-piercing tip 108 configured to pierce through the walls of the atrial appendage 2 during installation. Spike 106 may be rigid, or semi-rigid, such that it may be deformed by hand, but retains enough column strength to pierce the tissue walls by application of force to base 110. Examples of materials from which spike 110 may be made include, but are not limited to, polypropylene and nylon. Base 110 may be rectangular, circular or any other shape that provides a broad surface area for abutting the surface of a first wall of tissue 2, thereby preventing pull-through of the base 110 as the tissue walls 2 are placed under compression.

A collar 112 is provided to cooperate with spike 106 to compress the tissue walls. Collar 112 is substantially rigid and, like base 110, may be rectangular, circular or any other shape that provides a broad surface area for abutting the surface of a second wall of tissue 2, thereby preventing pull-through of the collar 112 as the tissue walls 2 are placed under compression. Spike 106 is further provided with ratchet features 114 on at least the proximal portion of the shaft of spike 106. Ratchet features each have a distal end having an outside diameter approaching or equal to the outside diameter of shaft 107 of spike 106 and a proximal end having an outside diameter larger than the outside diameter of the proximal end. Collar 112 has an inside diameter slightly larger than the outside diameter of shaft 107 and is also slightly larger than the outside diameter of the distal ends of ratchet features 114, but smaller than the outside diameter of the proximal ends of ratchet features 114. At least the distal end portions of ratchet features are sufficiently elastically deformable to be passed through collar 112 during installation of device 10, such that they return to their original configuration after passing through collar 112 and are thereby preventing from passing back through collar 112 in the opposite direction. Alternatively, the ratchet features may be made rigid and the collar 112 made of an elastically deformable material that is deformed as the rigid ratchet features pass therethrough. After a distal end of a ratchet feature passes through collar 112, the elastically deformable material returns substantially to its initial undeformed configuration, thereby preventing the distal end of that ratchet feature from passing back through collar 112 in the opposite direction of travel.

To install device 10, spike 106 is aligned adjacent a first wall of atrial appendage 2 at a target site where the walls are desired to be joined. Collar 112 is aligned with spike 106 adjacent the opposite wall of tissue. Axial force on spike 106 (such as applied through base 110, for example) causes tip 108 to pierce through both tissue walls 2 as tip 108 is inserted through collar 112. Spike 106 is advanced through collar 112 until walls 2 are sufficiently compressed to stop fluid flow therebetween, but not over-compressed to an extent that would cause tissue necrosis. The provision of a series of ratchet features 114 allows the compressive force to be adjusted to a level deemed to be optimal by the installer. An installed device 10 is shown in the sectional illustration of FIG. 7B, with walls 2 having been compressed to prevent fluid flow therepast. A portion of spike 106 that extends past collar 112 may be removed, such as by cutting it off. The proximal end of the ratchet feature that abuts collar 112 maintains device 10 in compression against walls 2, as the proximal end is prevented from passing back through collar 112 as described above.

Devices 10 may be installed adjacent one another, as close as desired, up to as close as an arrangement where adjacent collars 112 abut one another. Alternatively, devices 10 may be spaced apart slightly by a distance as determined sufficient by a surgeon performing the procedure. Devices 10 may be installed using a pliers-like tool configured to hold spike 106 and collar 112 as they are compressed together using the jaws of the tool, similar to the manner shown in FIG. 5A, but wherein the jaw holding collar 112 has a through hole to allow the tip of spike 106 to pass therethrough.

FIG. 7C illustrates another device 10 for closing together tissue walls and an apparatus used to install such device 10. In this example, device 10 includes a barrel shaped, rod-shaped or cylindrical base 116 which is sometimes also referred to as a “T-bar”. A flexible stem 118 extends proximally from base 116. Stem 118 is bendable to allow base 116 to be rotated to be slid within a delivery tube or needle 120 as shown in FIG. 7C. Tube or needle 120 may be slotted 122 at least at a distal portion thereof, as shown in FIG. 7D, to facilitate ejection of base. Tube or needle 120 may include slot 122 along the full length thereof to allow it be removed from stem 118 after ejection of base 116 before cutting or trimming the length of stem 118.

Stem 118 extends proximally from base 116 and after, insertion of base 116 into tube 120, has sufficient length to extend proximally from tube 20 as shown in FIG. 7C. Even after ejection of base 116 from tube 20, described below, stem 118 may have sufficient length to extend proximally from the proximal end of tube 20 when base 116 is outside of and near or adjacent to the distal end of tube 20. Collar 112 is mounted over a proximal portion of stem 118 to be slid further distally to compress the tissue walls 2 upon installation, as will be described below. A driver 124 is also threaded over stem 118 proximally of collar 112 and is slidable over stem 118 and against collar 112 to push collar 112 to cooperate with ratchet features 114 located further distally on stem 118. Collar 112 and ratchet features 114 cooperate in the same way as described above with regard to the device described with respect to FIGS. 7A and 7B, to lock collar 112 and base 116 against opposite walls 2 of tissue to hold them under compression to perform the ligation. A fixed member 126 is provided on stem 118 proximally of driver 124 and is fixed with respect to stem 118. A second driver 128, such as a rigid shaft or rod is provided to, upon advancement distally with respect to needle 120, abut base 116 and eject it from the distal end opening of needle 120. Second driver 128 has an outside diameter or periphery that is significantly less than the inside diameter or circumference of needle 120, to allow advancing second driver 128 therein without disrupting stem 118.

To install device 10, device 10 is loaded into the apparatus as shown in FIG. 7C. The apparatus is then advanced into the patient and the distal tip of needle 120 is aligned with target site 3 at the base of the atrial appendage where the ligation is to be performed. Needle 120 is inserted through both walls of the atrial appendage starting from the target site 3. Upon emerging from the opposite wall, second driver 128 is next advanced distally with respect to needle 120 to abut base 116 and then drive (eject) the base out of the distal end opening of needle 120. Upon emerging from needle 120, base 116 resumes its unstressed orientation as stem 118 straightens out so that stem 118 now extends substantially perpendicular to base 116. The operator may retract stem 118 slightly at this time by pulling on fixed member 126, for example, to draw base 116 into abutment with the opposite side wall of the atrial appendage 2.

Next, driver 124 is advanced distally with respect to stem 118 while preventing distal advancement of stem 118 by holding fixed member as driver 124 is advanced. Driver 124 is advanced (e.g., by hand) to engage ratchet members 114 and distal advancement is continued until collar 112 and base 116 have compressed the walls of the atrial appendage 2 sufficiently together to prevent blood flow past the site of the ligation, with care being taken not to over-compress the tissue walls, to prevent necrosis. Slot 122 of needle 120 is continuous over the length of needle 120, so that stem 118 can be passed therethrough (or needle 120 can removed from stem 118 via slot 122), thereby releasing the delivery apparatus from the implanted device. The excess length of stem 118 extending from collar 112 may be cut off, using scissors or other cutting instrument, for example,

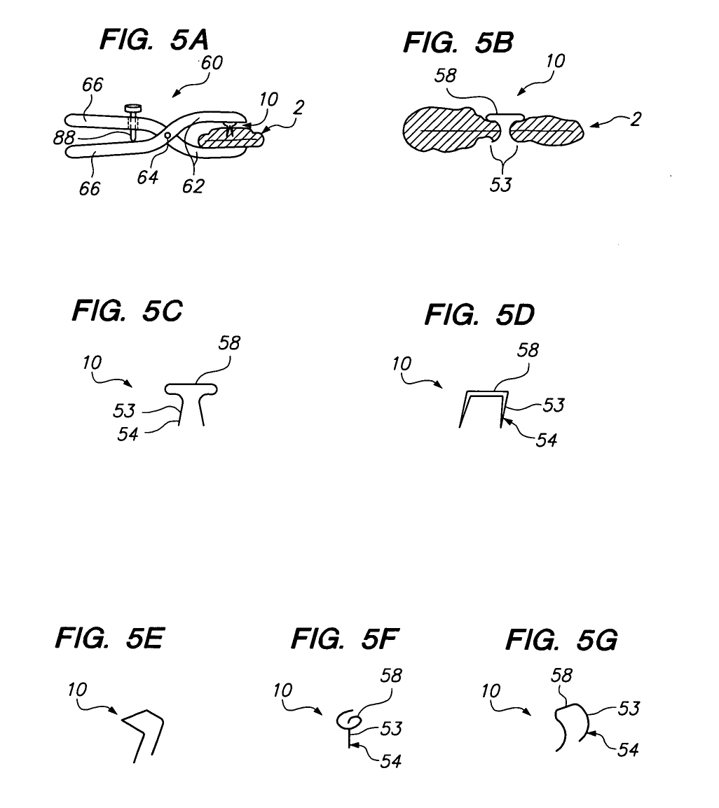

FIG. 8A shows another example of a device 10 useful for ligating an atrial appendage by holding the walls of the base of the atrial appendage sufficiently in apposition to prevent fluid flow therebetween. Device 10 includes two cooperating bodies or plates 130,132, which may be equally dimensioned, and which are configured to be drawn together to sandwich the walls of an atrial appendage 2 therebetween under compression to prevent blood flow into and out of the atrial appendage 2. Plates 130,132 may be rigid, or alternatively one or both may be malleable so as to be shaped to conform to the anatomy of the atrial appendage base for an optimum fit. Still further, device 10 may be made entirely or partially of one or more bioabsorbable materials to remove any potential of long term negative effects of maintaining an implant at the site.

Connecting members 134 are provided as sutures or flexible wires joined to a first plate (e.g., plate 130 in the example shown) and threaded through passageways in the other plate (plate 132 in the example shown) in a manner such that connecting members are slidable with respect to the plate (e.g., plate 132) that they are threaded through.

To perform a ligation, plates 130,132 are separated from one another to assume an open configuration, providing a gap between plates 130,132 sufficient to allow plates 130,132 to be passed over opposite tissue walls 2 to be ligated. Plates 130,132 are then oriented adjacent the opposite walls of tissue at a desired target site to perform the ligation. While holding at least the plate through which connecting members 134 are threaded to prevent movement of such plate (the other plate may optionally be stabilized in the same way) directions along the surface of the tissue wall (but still allowing movements of the plates 130,132 toward one another to effect compression), tension is applied to connecting members 134 thereby drawing plates 132 and 130 together in compression. Tension is applied until a sufficient degree of compression is placed on the opposite tissue walls to close off the space therebetween, thereby preventing fluid flow therebetween, but not so great as to strangulate the tissue 2. Connecting members may then be knotted or otherwise fixed together, thereby holding plates 130,132 in compression against the walls of tissue. Any excess suture or wire material existing proximal of the knots or other connection of connecting members 134 may be removed, such as by cutting for example.

FIG. 8B illustrates a variation of the device 10 described above with regard to FIG. 8A. In this arrangement, side walls 136,138 extend from plates 130,132, respectively, and are configured to act as a stop mechanism to limit the minimum gap that can be achieved upon drawing plates 130,132 together, as walls 136,138 abut each other and prevent plates 130,132 from being drawn any closer. Thus, the combined length of wall 136 and wall 138 defines the minimum gap that can exist between plates 136,138. Lengths of walls 136,138 can be predetermined to optimize the degree of compression that will be applied to walls 2 by plates 130,132 upon installation, and may define gaps having predetermined dimensional ranges as discussed previously with regard to earlier described devices. More generally, optimum wall lengths are dependent upon the thicknesses of the tissue walls 2 to be compressed. As such, a kit of devices having various wall 136,138 lengths may be provided, from which a surgeon may choose to deliver optimum compression to the walls, based upon a measurement of wall 2 thicknesses, for example.

FIG. 8C illustrates a variation of the device 10 shown in FIG. 8B, wherein device 10 in FIG. 8C has plates 130,132 with a curved contour along the lengths thereof to match the contour at the mouth (base) of an atrial appendage. The direction of curvature may be substantially perpendicular to the directions in which plates 130,132 are drawn together to abut walls 136,138. A kit of such devices 10 having different curvatures may be provided to allow a surgeon to select the device 10 which most closely approximates the contour of a particular atrial appendage base to be ligated.

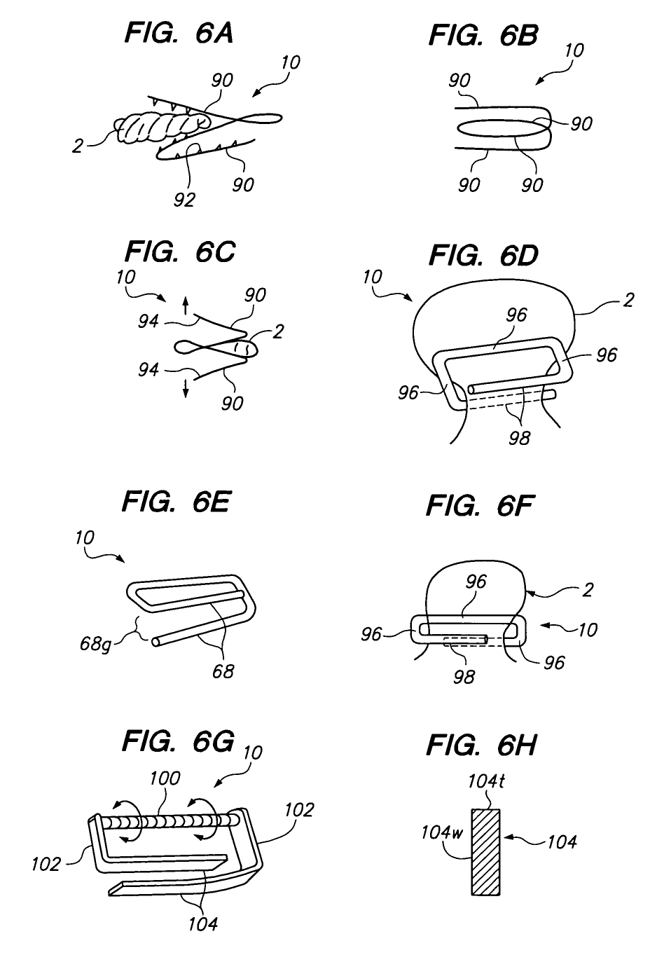

FIG. 9A illustrates a technique for atrial appendage ligation that may be practiced with any of the different devices described herein. After ligation at the base of the atrial appendage (i.e., target site 3), the atrial appendage 2 that extends from the base may be folded over and attached to other tissue on the atrium or other tissue nearby, thereby ensuring that atrial appendage remains in deflated, compressed configuration incapable of acting as a capacitance for a blood pool. Alternatively, FIG. 9B shows a technique wherein after ligation at the base of the atrial appendage (i.e., target site 3), the atrial appendage 2 that extends from the base may be twisted and folded over on itself and attached to the atrium or other nearby tissue to ensure that the atrial appendage remains closed. In either case, attachment may be accomplished by way or suturing, laser welding, adhesives, or by using an appropriate device 10 described herein.

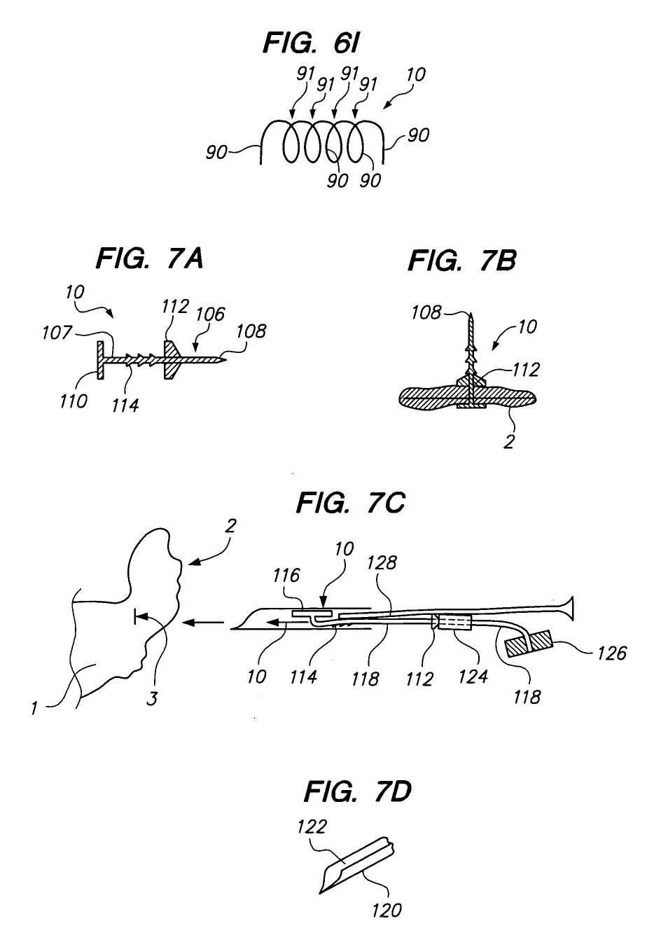

FIG. 10 illustrates a device 10 configured to ligate the base of an atrial appendage internally. In this example, an inflatable balloon 10 is inserted into the mouth of the atrial appendage 2 and inflated to expand the dimensions of the balloon to conform to the inner walls at the base of the atrial appendage and apply pressure thereto to form a fluid tight seal at the mouth of the appendage, thereby preventing fluid flow therepast.

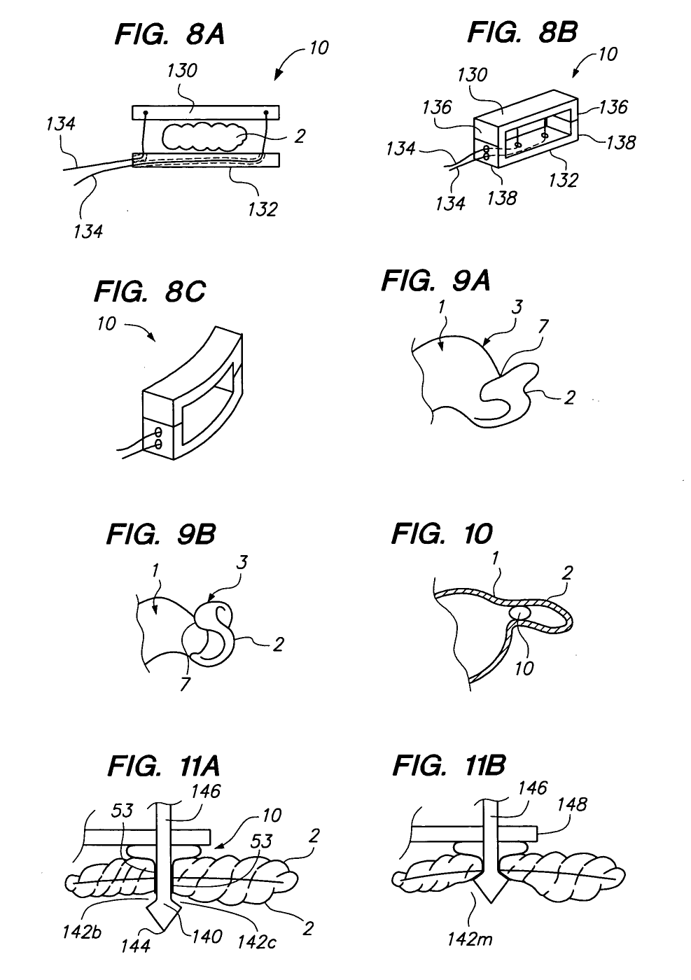

FIGS. 11A-11C schematically illustrate installation of another example of a device 10 for closing two walls of tissue together, particularly the walls at the base of an atrial appendage 2, wherein the tissue walls are shown as a sectional view in FIGS. 11A-11C. Device 10 includes malleable tines 53 (typically two, although more may also be provided, and a base 58 provided with sufficient area to provide leverage to device 10 in the clamped configuration to prevent base 58 from pulling through the tissue being clamped. The width or diameter of base 58 may typically range from about ⅛ inch to ¼ inch, with a typical width or diameter being about 3/16 inch. Device 10 is typically made of metal such as stainless steel or other biocompatible, malleable metal (e.g., thin wire or sheet metal), but may also be made from malleable polymers or composites that are biocompatible.

A spreader or anvil member 142 may be inserted axially through device 10 as shown in FIG. 11A, and is used to deploy device from an initial configuration (shown in FIG. 11A) to a final, clamping configuration shown in FIG. 11C. Anvil member 142 includes a pointed or otherwise sharpened distal end 144 that is adapted to pierce through the tissue walls 2 to facilitate the initial insertion of device 10 through the walls 2 as shown in FIG. 11A. The distal end portion of anvil member 142 tapers outwardly 142b from the distal tip 144 towards a portion 142m of the distal end portion having the largest cross-sectional area, and then tapers inwardly 142c from portion 142m to the proximal portion 146 of anvil member 142. Proximal portion 146 may be a slender shaft or cylindrical rod having an outside diameter smaller than an inside diameter of an opening through device 10, permitting proximal portion 146 to slide freely therethrough.

Enlarged portion 142m has an outside diameter sufficient to deform tines 53, to spread them apart and bend them into a clamping configuration as shown in FIG. 11B as anvil member 142 is retracted relative to the basel 48 of the delivery tool, that is anvil member 146 is drawn in the direction of the arrow shown in FIG. 1B, while base 148 is maintained in its position against tissue wall 2. Anvil member 142 may be drawn in the direction of the arrow in FIG. 11B using any appropriate mechanical means that provides sufficient force to draw the anvil through the device 10, including, but not limited to mechanical prying mechanisms, forceps, graspers or other mechanism specifically designed to accomplish this task, which would be readily apparent to one of ordinary skill in the art. The ramped portions of tapered portion 146c guide tines 53 outwardly, at the same time deforming them into the spread configuration dictated by 142c and 142m. Continuing the drawing force on anvil member 142 draws enlarged portion 142m though device 20, further separating tines 53 and compressing them against the lower tissue wall 2. The opening 58o in base 58 has a diameter larger than the outside diameter of enlarged portion 142m so that when enlarged portion 142m abuts base 148, the delivery assembly may be removed from the site, with enlarged portion 142m freely passing though opening 58o in base 58.

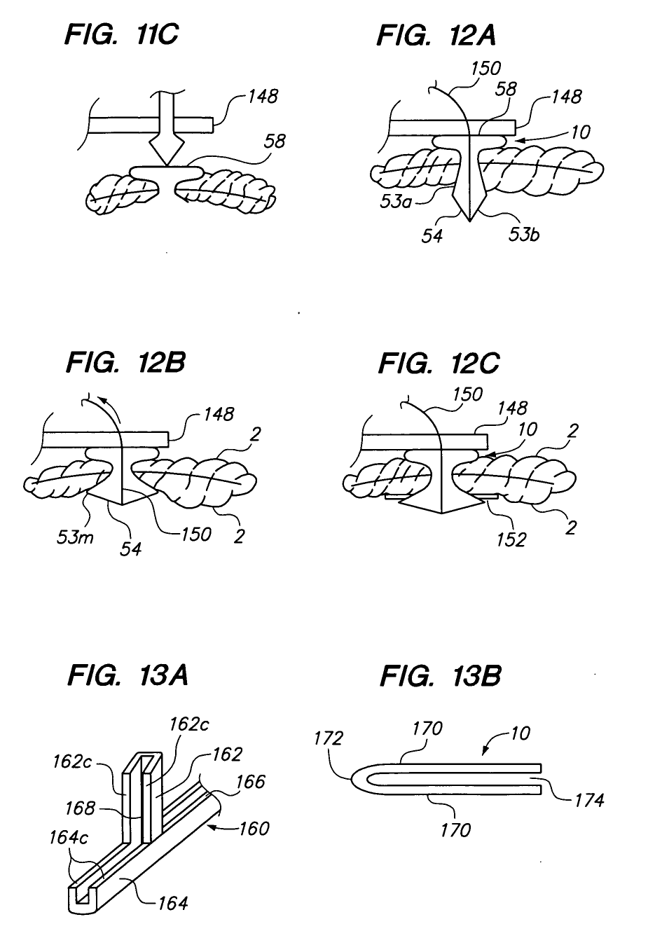

FIGS. 12A-12B schematically illustrates closing two walls of tissue together, using another variation of a device 10, wherein the walls are shown in sectional views in FIGS. 12A-12B. Device 10 includes malleable tines 53 (two or more) that extend from base 58 and join at a distal end to form a pointed or otherwise sharpened tip 54 configured to pierce through the tissue walls 2 as shown in FIG. 12A. Tines 53 are configured to buckle when device 10 in compressed along the longitudinal axis of device 10.

Tines 53 may be tapered similarly to the tapered shape discussed above with regard to anvil member 142. That is, tines 53 may taper outwardly 53a from a smaller cross-sectional configuration near base 58 to an enlarged portion 53m of the tines having the largest cross-section of the portion of device 10 formed by tines 53. Further, tines 53 may taper inwardly 53b from enlarged portion 53m to tip 54. Tapering 53b facilitates the insertion of tines 53 through tissue 2 as led by tip 54. Tapering 53a facilitates compression against tissue 2 during deployment (compression) of device 10. Device 10 may be made from any of the materials discussed above with regard to device 10 described in FIGS. 11A-11C.

A tensioning member 150 extends longitudinally through device 10 and is fixed at its distal end to tip 54, internally of device 10. The proximal portion of tensioning member 150 extends proximally of device 10 and is of sufficient length to extend out of the body of the patient so as to be manipulated by an operator (surgeon) from outside the body. Maintenance of a slight to moderate tension on tensioning member 150 maintains base 58 contacted against tool/base 148 during installation of device 10. Additionally or alternatively, device 10 may be temporarily mechanically or chemically fixed to tool base 148. Tensioning member 150 may be provided as a wire, suture or other string-like material having sufficient tensile strength to deform the device 10 in the manner described.

Once device 10 has been inserted through tissue walls 2 as shown in FIG. 12A, such as by driving device 10 using tool 148, additional tension is then applied through tensioning member 150, proximally with respect to tool 148, while maintaining tool 148 relative stationary, in contact with tissue 2. Tension is applied with sufficient force to buckle tines 53 in the location of enlarged portion 53m, e.g., where tapered portions 53a and 53b meet. This causes compression of device 10, as tapered portion 53a drives against tissue 2 thereby compressing the tissue walls between tapered portions 53a and base 58, as shown in FIG. 12B. Compression is continued until a predetermined force has been achieved or until confirmation that the tissue walls have been sufficiently compressed, as determined by measurement, or by visual observation and the judgment of the operator performing the procedure. Consequently, the tissue walls have been closed together to prevent blood flow therethrough. Tensioning member 150 can then be cut (e.g., either proximal of tool 148, or tool 148 can be moved proximally along tensioning member 150 so that tensioning member 150 can be cut immediately proximal of base 58) and tool 148 as well as the proximal portion of tensioning member having been cut off are removed from the site.

FIG. 12C shows a variation of the procedure described above with regard to FIGS. 12A-12B. In this example, a washer 52 of other member is provided to increase the surface area over which the compression force is applied to tissue 2. In the example shown, washer 52 is placed over tip 54 of device 10 and abutted against tissue 2 when device 10 is at the stage of installation as shown in FIG. 12A (i.e., not yet compressed). Upon compression, in a manner as described with regard to FIG. 12B, the expanding portions 53a are drawn against washer 152 which in turn compresses tissue 2. Alternatively or additionally to provide a surface area enhancement member 152 as described with regard to FIG. 12C, a surface area enhancement member 152, such as a washer for example, may be placed over tip 54 and tines 53 prior to insertion of device 10 through tissue walls 2 to enhance the surface area over which compression forces are applied at the base 58 end portion of device 10.

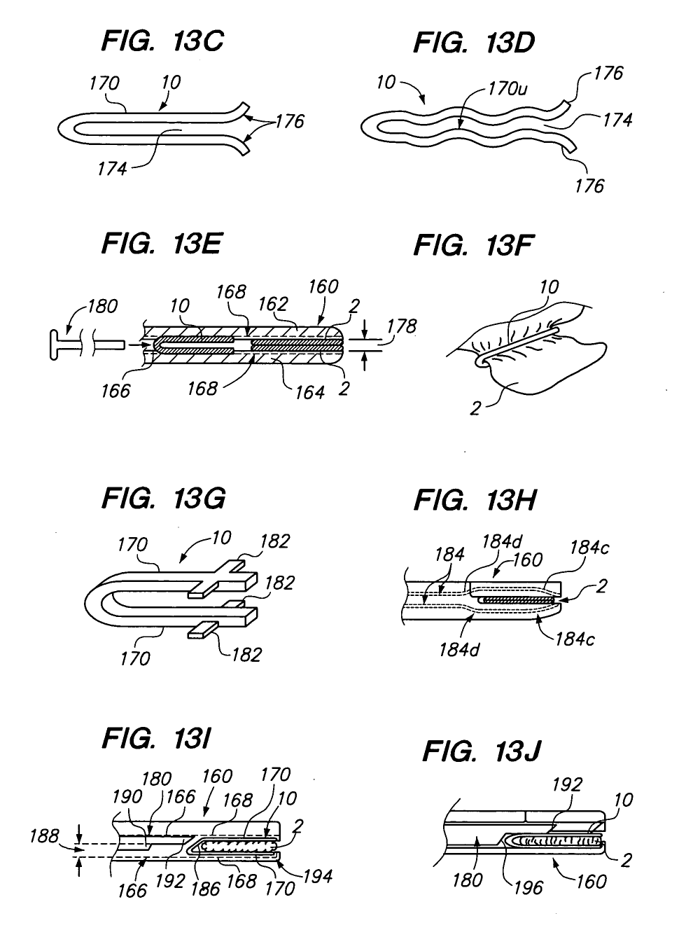

FIG. 13A is a partial perspective illustration of a tool 160 configured for installation of a device 10 (such as any of the devices 10 shown in FIGS. 13B-13D, for example) over opposing walls of tissue 2 to ligate the tissue to prevent blood flow between the opposing walls as ligated. Opposing jaws 162 and 164 are mounted for articulation with respect to one another, such as by pivot 166, for example, or other joint, so that jaws can be driven together to compress tissue walls 2 therebetween in preparation for a ligation. A channel 166 passes through the entire length within tool 160 and is dimensioned to allow device to be easily slid therethrough for delivery via an opening at the proximal end of tool 160 to tissue walls as clamped by the distal end portion of tool 160 where jaws 162,164 are formed. Grooves or channels 168 in jaws 162,164 meet to extend channel 166 to the distal end of tool 160 when jaws are approximated together.

Device 10 comprises a clip that may rigid, and made from a biocompatible plastic, metal or composite, or alternatively may be made from a biocompatible spring steel or other metal or plastic that provides arms 170 with elastic, spring force. Device 10 includes a pair of longitudinally extending arms 170 that extend substantially parallel to one another and are integrated at their proximal ends forming joint 172. Arms 170 are provided with a length to span the distance of the tissue to be ligated. As such, for purposes of ligating an atrial appendage, arms 170 may have a length at least equal to and typically slightly greater than the width of the atrial appendage 2 in a location where ligation is to be performed. Since widths of atrial appendages can vary from patient to patient, a kit of devices 10 may be provided having varying arm lengths 170.

A predefined gap 174 may be formed between arms 170 having a dimension designed to receive tissue walls 2 after having been compressed together by tool 160 as will be described below. Since wall thicknesses of tissues can vary (even among tissues of the same type, such as atrial appendage walls, when comparing different patients), a kit of devices 10 may be provided having varying gap distances 174, with or without varying arm lengths 170. The distal ends of arms 170 may be angled apart or fluted 176 to facilitate the reception of tissue walls 2 between arms 170. Further, arms 170u may be formed to ripple or undulate to enhance friction between arms 170u and tissue walls 2 for providing further assurance that the compressed tissue 2 will not slip out from the grasp of arms 170u.

FIG. 13E illustrates a portion of a procedure for ligating an atrial appendage 2 using device 10 as shown in FIG. 13B and tool 160. In this example, tool 160 has been advanced over an atrial appendage at the desired location where the appendage is to be ligated, while jaws 162,164 are at least somewhat opened apart from one another to facilitate positioning of the jaws. Jaws 162,164 are then approximated together, as shown in FIG. 13E to clamp down on the tissue walls 2, thereby clamping them together. Jaws 62,64 may be configured with a stop so that they cannot be closed completely together in contact with one another, but are stopped when a predefined gap 178 has been defined. Predefined gap 178 may be substantially equal to gap 174 described above, or may be slightly less than 174. Device 10 may be made from any of the materials described above with regard to device 10 described in FIGS. 11A-11C. Predefined gap 178 is defined by the clamping surfaces 162c,164c of jaws 162,164, and not grooves 168. As noted earlier, grooves 168 extend channel 166 and guide the passage of device 10 over tissue walls 2. A pusher 180 such as a rigid rod, shaft or other elongated, slender rigid member that may be easily slid within channel 166,168 and will not buckle under the compression forces required to advance clip may be inserted into channel 166 at the proximal opening of tool 160 and advanced distally to contact device 10 and push it into position over tissue walls 2. Once device 10 has been fully positioned over tissue walls 2 so as to hold them in their clamped position, pusher 180 may be removed and jaws 162,164 may be unclamped from tissue walls 2 after which tool 160 may be removed from the site. FIG. 13F is a perspective view illustration of an atrial appendage 2 having been ligated by a device 10 according to the procedure just described. FIG. 13F shows the completed ligation, with device 10 shown completely clamping off the atrial appendage 2 to prevent blood flow thereto.

FIG. 13G shows a variation of device 10 that includes tabs or other extension 182 extending laterally from one or both sides of arms 170. Tabs 182 may be integrally formed with arms 170 of the same material. Tool 160 may be provided with grooves or channels 184 as shown in FIG. 13H to guide the travel of tabs 182 therethrough. Channels 184 receive tabs 182 and guide tabs 182 and thus the degree of opening of arms 170, relative to one another, during the installation of device 10. Channels 184 diverge from the central longitudinal axis of tool 160 at 184d toward the distal end portion of tool 160, as shown in FIG. 13H, to drive the distal ends of arms 170, via tabs 182, open or apart to ensure that arms 170 clear the tissue 2 as device is passed over the tissue. Further distally, channels 184 converge toward the central longitudinal axis of tool 160 at 184c to guide tabs 182 and arms 170 together, so as to clamp down against the tissue 2. Arms 170 are elastically deformable and spring back to the position shown in FIG. 13G to complete the clamping of the tissue walls upon installation.

FIGS. 13I-13J illustrate another variation of performance of a ligation of an atrial appendage using tool 160 and clip 10. In this variation, device 10 is made of a material that is sufficiently rigid to maintain the tissue walls clamped shut upon completion of the ligation and removal of tool 160, yet sufficiently malleable to be deformed by pusher 180 as described below. Device 10 may be formed with a beveled or angled (with respect to a perpendicular to the longitudinal axis of tool 160 when device is guided through channels 166,168) proximal end 186 with arms 170 extending distally therefrom. The distal ends of arms 170 may be straight, as shown, or, optionally, may be angled apart or fluted (like 176 shown in FIG. 13D) to facilitate the reception of tissue walls 2 between arms 170. Initially, the gap between arms 170 is sufficient to receive tissue walls 2 therebetween without imposing compression thereagainst. Since widths of atrial appendages or other tissue walls to be ligated can vary from patient to patient, a kit of devices 10 may be provided having varying arm lengths 170. Additionally or alternatively, since wall thicknesses of tissues can vary (even among tissues of the same type, such as atrial appendage walls, when comparing different patients), a kit of devices 10 may be provided having varying gap distances between arms, with or without varying arm lengths 170. Such a kit may also be provided with pushers 180 having varying compressed dimension gaps 188, the functions of which are described below, and/or tools 160 having varying gaps between channels 168.

The body or main shaft 190 of pusher 180 is dimensioned to ride in channels 166 to maintain alignment of the distal end portion of pusher 180 with device 10. A first pushing surface 192 is formed at the distal end of pusher 180. First pushing surface may be beveled or angled to match the angle of the proximal end portion of device 10 as shown. Once tool 160 have been positioned over the tissue walls 2 and jaw 162 has been closed and locked (using any conventional locking mechanism, which may be readily apparent to one of ordinary skill in the mechanical arts) as shown in FIG. 13I, pusher 180 is advanced distally with respect to tool 160 so that distal end 192 contacts proximal end 186 of device 10. Continued advancement of pusher 180 in the distal direction pushed device 10 distally as it slides through channels 168. When device 10 has been positioned over the tissue wall, in the location desired to perform the ligation, the distal end of lower arm 170 abuts against a stop 194 formed in channel 168 of tool 160, thereby preventing any further advancement of device 10 in the distal direction with respect to tool 160.

Continued advancement of pusher 180 causes distal end 192 to deform the proximal end of malleable device 10 as shown in FIG. 13J, thereby compressing device 10 against tissue walls 2 in performance of the ligation. The difference in thicknesses 188 between the main body 190 and the distal end portion of pusher 180 may be predefined as the desired combined thickness of the tissues 2 and arms 170 in the compressed configuration that accomplishes the ligation, as shown in FIG. 13J. Pusher 180 may be provided with a secondary end surface 196 that prevents backsliding of device 10 during compression. Secondary end surface may be proximal of first pushing surface by a distance approximately equal to the length of device 10, or may be provided at a shorter distance, as the primary deformation and compression forces are provided at the proximal end portion of device 10 during performance of the ligation. As shown in FIG. 13I, the proximal end portion of device 10 is provided with an acute angle and an obtuse angle. During deployment, the acute angle is deformed to a smaller angle. As the acute angle is reduced, the distal ends of device 10 close towards one another and the obtuse angle thereby increases, As the obtuse angle increases, this provides the distal end portions of device 10 with a closing spring force against the tissue walls 2. After completion of the compression as described, tool 160 and pusher 180 are removed, leaving device 10 in place, thereby completing the ligation.

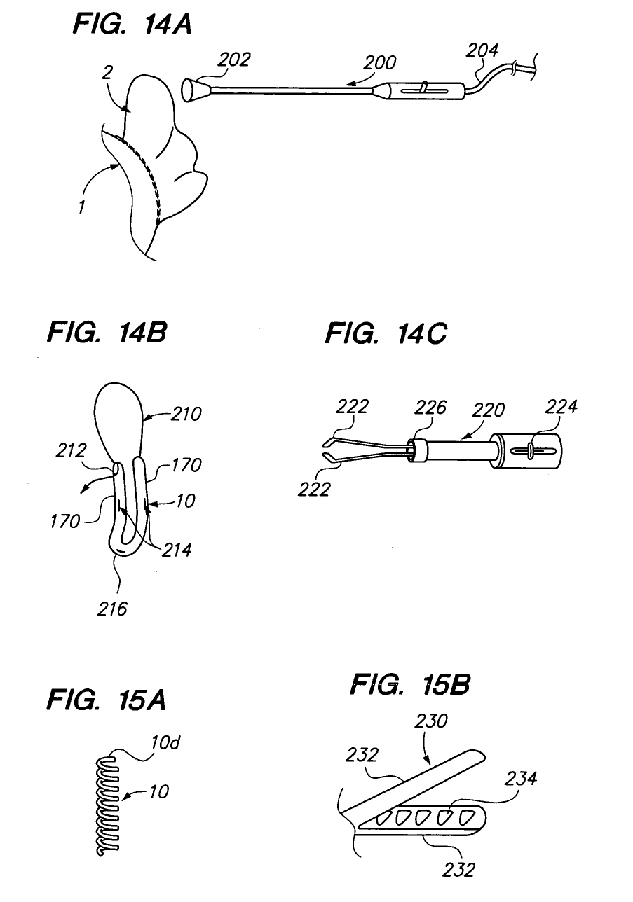

Turning now to FIGS. 14A-14C another arrangement of tools and device are shown for performing a ligation, particularly for ligating an atrial appendage. Tool 200 is a suction applicator that may be inserted through a small opening to apply suction to tissue 2 to be ligated, to stabilize the surgical site during performance of the ligation. A suction cup or other tissue contacting member 202 capable of applying negative pressure to the tissue to form a seal therewith is provided at the distal end of tool 200 and is fluidly connectable through tool 200 and proximally out of tool 200 via suction conduit 204, to a source of negative pressure (not shown), such as a vacuum source that is typically provided in a surgical operating environment, for example. Tissue contacting member 202 may have a diameter or width of about ¼ inches to 2 inches, for example, and may be made of biocompatible elastomer or rubber, for example. Upon contacting tissue 2 with suction member 202, negative pressure is applied to tissue 2 to form a seal between tissue 2 and suction member 202, thereby fixing the suction member 202 to the tissue 2. Tool 200 can then be manipulated to move/position the tissue 2 as desired, as well as to steady the tissue when performing the ligation.

Device 10 includes arms 170 and is configured similarly to devices described previously. In this example, however, device 10 is typically formed of a nickel-titanium alloy or other shape memory material that retains a memory of the compressed configuration of device 10. A flexible tie line 210 may also be provided, which is fixed to a distal end portion of one of arms 170 and is threaded through an opening 212 through a distal end portion of the other of arms 170. Tie line may be made of an elastic silicone material, suture material, or the like, for example. Device 10 further includes slots or other engagement features 214 on the arms thereof for engagement by tool 220 that is used to spread the arms 170 of device 10 open during placement of device 10 over the tissues to be ligated.

To perform the ligation using the arrangement of FIGS. 14A-14C, tangs 222 of tool 220 are engaged with slots 214 of device 10 and actuator 224 is moved toward the proximal end of tool 220 to slide collar 226 proximally with respect to tool 220, thereby allowing spring biased members 228 to expand or move apart from one another, thereby also separating tangs 222 and spreading open the arms 170 of device 10. Device 10 is then maneuvered, using tool 220 and passed over the tissues to be ligated. In this example, device 10 is passed over the atrial appendage 2, so that tie line 210 and arms 170 surround the appendage 2. Suction may then be applied through tool 200 to engage tissue 2 so that the tissue can be manipulated as well as device 10 to properly position device 10 to perform the ligation. Alternatively, tissue 2 may be engaged by suction member 202 prior to placement of device 10, as device 10, in the expanded configuration described, can be passed over tool 200 and then over the tissues 2 to be ligated.

In either case, once device 10 surrounds tissue 2, device 10 is then positioned so that arms 170 traverse the tissues to be compressed, overlying the target area where the ligation is to be performed. Actuator 224 is then moved distally with respect to tool 220, moving collar 226 in the same direction and bringing tines 222 toward one another, thereby clamping the tissues 2 and compressing them with sufficient force to ligate the atrial appendage. Once the tissues 2 are clamped as described, tie line 210 may then be drawn through opening 212 until tie line 21 abuts tissue 2, thereby, together with arms 170 and the proximal end of device 10, completely encircling the tissues 2. Tie line 210 may then be knotted or provided with an anchor to prevent tie line from loosening by passing back through opening 212 in the opposite direction. Thus tie line 210 provides further assurance that device arms 170 will not slide back or become partially or totally displaced from the target area intended, and may also ensure that the intended compression forces are maintained by arms 170 against the tissue walls 2.

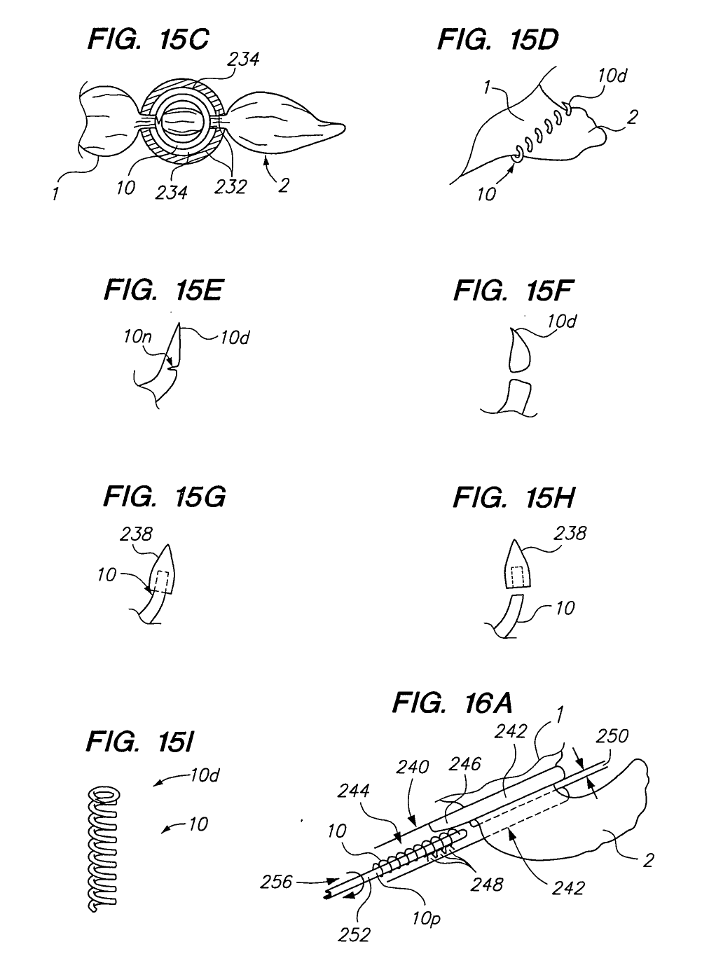

FIG. 15A shows another variation of a device 10 configured to maintain tissue walls compressed together to prevent blood flow therebetween. In this arrangement, device 10 is formed as a spiral device having an inside diameter preset to a desired thickness of the tissue walls when under compression to perform a ligation. Device 10 may be made of spring steel, nickel-titanium alloy, or other material having sufficient elasticity and spring force to maintain tissue walls under compression. Guide tool 230 (see FIG. 15B) is provided to clamp the tissue walls 2 under compression and to guide the installation of device 10. Jaws 232 of tool 230 are provided with pockets or indented guides 234 that function as anvils or guides against which the end and loops of the spiral of device 10 ride during installation of device 10.

FIG. 15C is a sectional view of tool 230 having been clamped over tissues to ligate an atrial appendage 2. Tool 230 may be provided with a stop so that when jaws 232 are clamped over the tissues, the clamping action stops at a predetermined gap between the inner surfaces of jaws 232 so that tissue walls 2 are held under sufficient compression to prevent blood flow therebetween, but not under so great a compression as to cause necrosis. Once tool 230 is clamped in the desired location, device 10 is then wound in through the proximal end of tool 230, distal end 10d first. As the coils of device 10 wind through pockets 234, end 10d pierces through the top and bottom walls, then bottom and top walls and repeats the cycle, or vice versa, depending on which wall is pierced first, as the device 10 winds its way through guide tool 230. The wall 236 of the distal most pocket 234 may act as a stop that distal end 10d abuts when it reaches stop 236.

Once device 10 is fully wound into position, jaws 232 are unlocked and opened, and tool 230 is removed leaving device 10 in place to complete the ligation as shown in FIG. 15D. One problem with this approach is that the distal end 10d of device 10 should be sharply pointed to pierce through the tissue walls 2 with ease during installation and with as little displacement of tissues as possible. This leaves the potential of tip 10d being exposed upon completion of the installation, which can harm surrounding tissues. One solution to this potential problem is to cut or break off the sharpened distal tip 10d if it protrudes upon completion of placement. Optionally, device 10 may be notched 10n or otherwise weakened just proximal of distal tip 10d to facilitate removal of distal tip 10, as shown in FIGS. 15E-F.

Another solution is to provide device 10 with a distal end cap 238 a having a sharp pointed end, as shown in FIGS. 15G-H. Distal end cap 238 is configured to form a friction fit with the distal end of coil 10, which is blunt, the friction fit having sufficient grip so that cap 238 cannot be displaced even if device has to be backed out or reverse-rotated for any reason. Upon successful completion of the installation of device 10 to perform the ligation, cap 238 can then be removed by pulling it off the end of device 10, such as by using graspers, or other surgical tool, for example.

A third solution is to manufacture device 10 so that distal end 10d forms a closed end with the adjacent coil of device 10 in the undeformed state as shown in FIG. 151. The elastic properties of device 10 allow distal end 10d to be separated from the adjacent coil as it is threaded into guide tool 230, and pockets 234 maintain the separation as tip 10d is passed through multiple layers of tissue walls 2 in the manner described above, as they guide tip 10d about the desired spiral pathway. When distal tip 10d is screwed beyond the distal end of guide tool 230, or when the jaws 232 are opened (as in the case where a stop is provided, for example) distal end 10d springs back into contact with the adjacent coil, thereby closing the sharpened tip against the adjacent coil, as it is in FIG. 151.

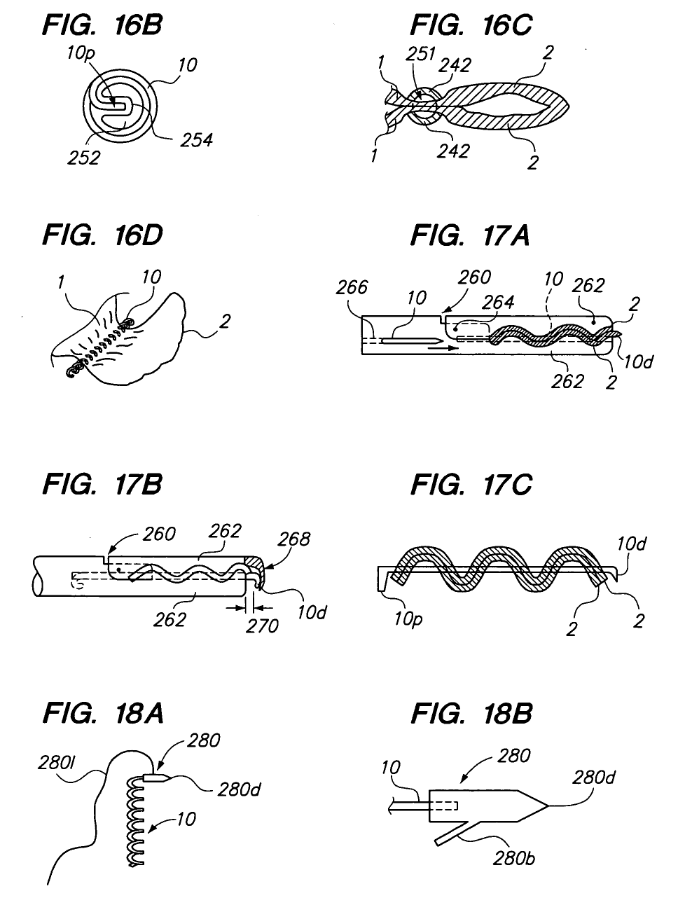

FIG. 16A illustrates another procedure for ligating an atrial appendage using a spiral-shaped device 10 configured to maintain tissue walls compressed together to prevent blood flow therebetween. Using a device 10 of a type such as shown in FIGS. 15A and 15I for example, device 10 may be installed using guide tool 240. Guide tool 240 includes main housing 244 and jaws 242 provided to clamp the tissue walls 2 under compression and to guide the installation of device 10. Upper jaw is pivotable via joint 246 to allow the jaws to be separated for initial placement over the tissue walls to be compressed, as well as to facilitate removal of tool 240 after device 10 has been placed. Both main housing 244 and jaws 242 are provided with protruding features 248 (only a small portion of which is illustrated) shaped like ACME threading or bosses, which function as threads that device 10 follows as it is threaded through tool 242. When jaws 242 are locked in the clamped configuration shown in FIG. 16A, they are separated by a predetermined gap 250 to maintain the tissue walls 2 under the desired degree of compression, as well as provide a circular cavity 251 for device 10 to travel in, as shown in FIG. 16C.

A rigid driver 252 is formed as a shaft or extrusion having a slot 254 running the length thereof. Driver 252 has an outside circumference or cross-sectional perimeter that is less than the inside diameter of device 10 to allow it to be inserted through device 10 as shown in FIG. 16A. Although shown as cylindrical, the cross-sectional shape of driver 252 need not be circular but could be of some other shape, including irregular shapes. The proximal end 10p of device 10 may extend inwardly with respect to the inside circumference defined by the inside diameter of the remainder of the coils of device 10, so that proximal end 10p is inserted into slot 254 to be engaged by driver 252 for torquing device 10 to thread it though the guide tool 240 during installation. FIG. 16B shows a proximal end view of driver 252, slot 254, device 10 and the proximal end 10p of device 10 inserted into slot 254.

By positioning driver 252 and device 10 as shown in FIG. 16A and rotating driver 252 in the direction of arrow 256 shown, device 10 is screwed into the tissue walls 2 between jaws 242. Upon completion of the installation of device 10 through the tissue walls 2, driver 252 is withdrawn proximally, jaws 242 of tool 240 are separated and tool 240 is removed, leaving the ligated appendage 2 as shown in FIG. 16D. Note that the same solutions for addressing the pointed distal end of device 10 may be applied in this situation as were discussed above with regard to FIGS. 15E-15I.

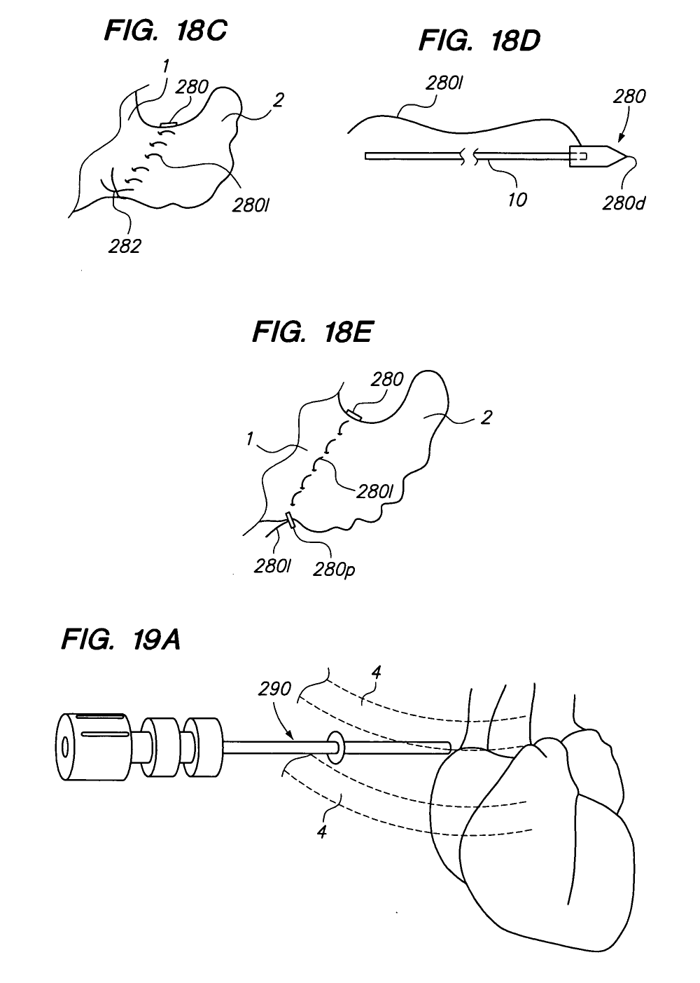

FIGS. 17A-B illustrate another technique and apparatus for joining tissue walls under compression to prevent blood flow therebetween. In both instances, a guide tool 260 is provided with jaws 262 having undulating or “wave-form” inner surfaces against which tissue walls to be joined are compressed. Upper jaw 262 is pivotable via joint 264 to allow the jaws to be separated for initial placement over the tissue walls to be compressed, as well as to facilitate removal of tool 260 after device 10 has been placed. Upon locking jaws 262 over tissue walls 2, tissue walls are compressed and conform to the undulating inner surfaces of jaws 262 as shown in FIG. 17A. A predefined gap is maintained between the undulating surfaces of jaws 262 in the locked configuration, to place the tissue walls under a desired degree of compression, without over-compressing them. Kits of tools 260 may be provided having varying lengths of jaws and/or predefined gaps between undulating surfaces to accommodate different lengths of tissue walls to be ligated, as well as different tissue wall thicknesses.

Tool 262 is provided with a channel 266 dimensioned to guide device 10. therethrough for the installation of device 10. Device 10 in this instance is a substantially straight, substantially rigid needle having a sharpened distal end 10d. Device 10 is advanced through channel 266 to pierce through (skewer) the undulations of the tissue walls 2, as shown in phantom in FIG. 17A. After such placement of device 10, jaws 262 are separated and tool 260 is removed, leaving tissues 2 in the compressed configuration as maintained by device 10 piercing therethrough. The sharpened tip 10d of device 10 may be treated by any of the techniques described above with regard to FIGS. 15E-15I to prevent damage to surrounding tissues after performance of the ligation.

Alternatively, the tool 260 of FIG. 17B is provided with a distal deflector 268 on one of jaws 262 (although deflector 268 is shown on the top jaw 262, it may alternatively be provided on the bottom jaw 262) that intersects the axis of channel 266. Thus, when distal tip 10d is driven against deflector 268 it is bent to follow the surface of deflector 268 which functions as an anvil. A gap 270 is defined between the anvil surface of deflector 268 and the distal end of the jaw 262 from which deflector 268 does not extend, to accommodate the thickness of the compressed tissue walls 2. A kit of tools 260 having varying gap 270 distances may be provided to accommodate varying tissue wall thicknesses. Additionally, the proximal end 10p of device 10 may be bent over (such as with graspers or other surgical instruments), as shown in FIG. 17C, after placement of device 10, to prevent damage to surrounding tissues.

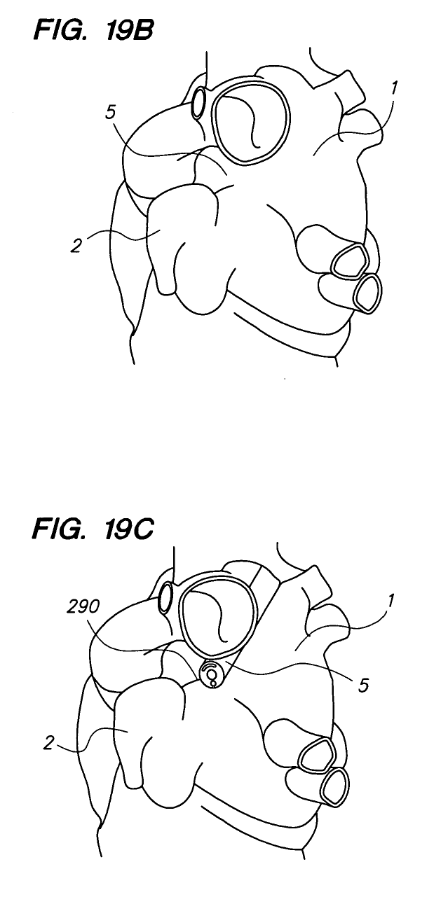

FIG. 18A shows a device that may be installed similarly to the techniques shown in FIGS. 15A-C, 15I and 16A, using the same tools described therein. Alternatively, device 280 may be implanted using a straight needle or driver according to the techniques described with regard to FIGS. 17A and 17B, wherein the needle is removed after installing the device 280. With this arrangement, an anchor 280 (such as a T-bar, for example) is mounted at the distal end of the spiral device (or straight needle) which is used to place anchor 280. Anchor 280 has a sharpened distal end 280d for facilitating the piercing of the tissue walls 2, and a flexible line 280l fixed to the main body of anchor 280 and having a sufficient length to span the tissue walls being ligated. Anchor 280 is substantially rigid and is temporarily fixed to the end of spiral 10 such as by sliding over the distal end of spiral 10 with a snug fit to keep anchor 280 from falling off during delivery, but loose enough so that when spiral 10 is withdrawn the proximal end of anchor 280 abuts against tissue wall 2 and remains abutted against the tissue wall, separating from the distal end of spiral coil 10. Optionally, anchor 280 may include one or more barbs 280b, as shown in FIG. 18b, that facilitate abutment against the tissue wall as spiral coil 10 is withdrawn. Barb(s) 280b may be elastically flexible, so as to deflect and substantially conform to the main body of anchor 280 as anchor 280 is driven distally through tissue walls 2, and then spring out into the configuration shown in FIG. 18B when barb(s) 280b has cleared the tissue walls, or, alternatively, barb(s) 280b may be rigid and formed in the configuration shown.

As noted, installation of anchor 280 through tissue walls 2 may be performed using the tools and techniques described above with any of FIGS. 15A-C, 15I and 16A. Once anchor as been passed through the tissue walls 2 at the distal extend of the ligation site, spiral 10 is then backed-out (i.e., reverse-rotated) to withdraw the spiral coil 10 form the tissue walls. Flexible line 280l at this time extends completely through the ligation site, through all the entry and exit holes in the tissue walls 2 and connects anchor 280, at one end of the ligation site, with the proximal entry site into the tissue walls. Flexible line 280l may then be tied off 282 to maintain tension thereon, thereby maintaining the tissue walls under compression, via the tie acting as a proximal anchor and the distal anchor 280 that abuts the tissue, as shown in FIG. 18C, or an additional anchor may be tied, friction-fitted, welded, or fixed by some other permanent fixation means to accomplish the same result. Jaws 232 or 242 may then be opened to remove tool 230 or 240. Tool 230 or 240 may be removed either before or after tying off line 280l.

FIG. 18D shows anchor 280 mounted to a straight needle 10, for installation using, in part, the techniques and tools described above with regard to FIGS. 17A-17B. After delivery of anchor 280 using such techniques and tools, needle 10 is withdrawn from the ligation site and flexible line 280l is tied off or fixed with an additional anchor 284 to maintain line 280l under tension, and thus tissue walls under compression, thereby completing the ligation. Jaws 262 may then be opened to remove tool 260. Tool 260 may be removed either before or after tying off line 280l. Like the previous example, anchor 280 may be provided with one or more elastically flexible or rigid barbs 280b. FIG. 18E shows a completed ligation according to that described in FIG. 18D, wherein the proximal anchoring has been performed by tying another T-bar type anchor 280p against the tissue wall 2 at the proximal end of the ligation.

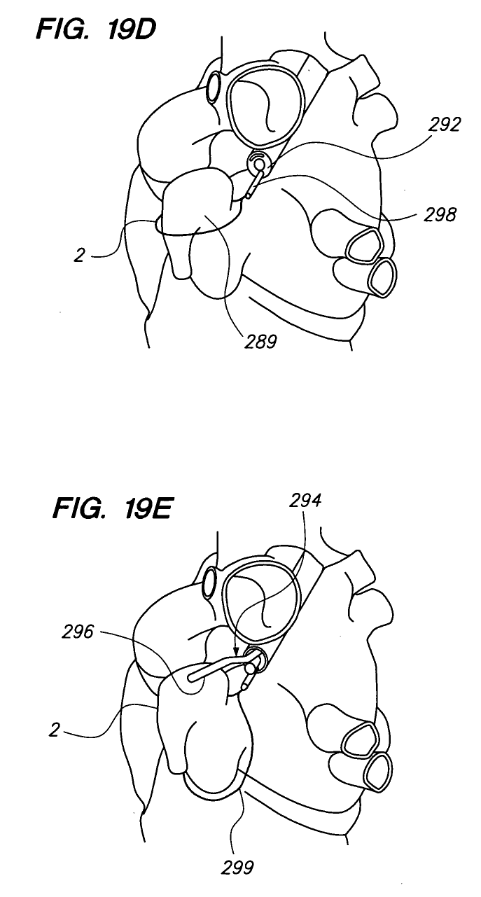

Turning now to FIGS. 19A-19F, another technique and associated apparatus for ligation of an atrial appendage in a closed-chest surgical environment is described. A multi-lumen endoscopic tool 290 is provided for access to an atrial appendage in a closed-chest environment, such as via a port or other small opening in the right or left chest of a patient, for example. FIG. 19A shows tool 290 having been inserted through a port located between ribs 4 in the right chest of a patient. Alternatively, access may be gained via a port or other small opening through the left chest, as already noted, or a sub-xyphoid port, for example.

After creating access to the target site, such as by preparing a port through the right chest as shown in FIG. 19A, for example, the multi-lumen endoscopic tool 290 is inserted through the transverse sinus 5 as illustrated in FIGS. 19B and 19C, viewing from the left side of the heart, where the distal end of tool 290 can be seen in the location of the transverse sinus 5 in FIG. 19C. Tool 290 is provided with a small diameter scope 292 (see FIG. 19F) which may have zooming and variable focal length and variable view angle features. A steerable suction tool 294 with a contact surface 296 having at least one suction opening connectable to a source of negative pressure outside the patient via a suction line extending greater than the length of tool 290 is inserted through one of the lumens in tool 290 for manipulation of the atrial appendage as will be described. A commercially available snare device 298 is inserted through another lumen of tool 290 to be used to snare the surgical site to be ligated. Alternatively, the snare device may be custom made to incorporate steerability. Additional lumens may be provided for insertion of other tools such as graspers, pushers or scissors, for example.

Once the transverse sinus has been located by viewing through scope 292, for example and the distal end of tool 290 has been inserted into the transverse sinus 5, tool 290, together with snare tool 298 are advanced to the location of the left atrial appendage and the snare 299 of snare tool 298 is looped over the left atrial appendage as shown in FIG. 19D. Suction tool 294 is then advanced to position contact surface 296 over the left atrial appendage 2 as shown in FIG. 19E and suction is applied to fix contact surface 296 to the distal part of the left atrial appendage 2. Suction tool may then be manipulated/steered to lift the left atrial appendage 2 in a direction away from the base of the left atrial appendage (the ligation site), thereby facilitating the position of snare 299 at the base of left atrial appendage, as shown in FIG. 19E. Ligation of the left atrial appendage is then performed by tightening snare 299 as shown in FIG. 19F. The tightened snare may then be locked in the tightened configuration and then removed from the snare device, such as by cutting for example, using endoscopic scissors inserted through another lumen of the multi-lumen device, for example.

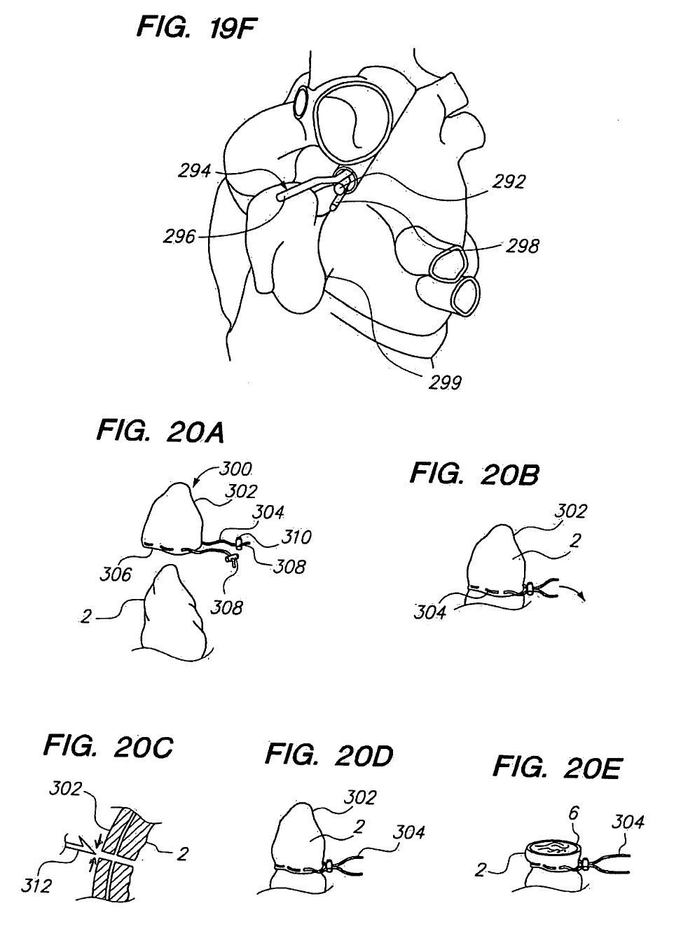

FIGS. 20A-20E illustrate a tissue wall coating device 300 and methods for use thereof in managing surgical procedures on tissues so coated by the device. Particular examples described are with regard to an atrial appendix, although device 300 may be configured for similar operability to coat other tissue structures. The main body 302 of device 300 may be formed as an elastomeric sack or cap, configured to form a tight, slightly compressive interface with the tissues that it surrounds.

A ligature 304 extends around a base portion of main body 302 and is arranged to reduce or constrict the opening 306 in the main body by drawing on one or both ends of ligature 304. In the example shown, ligature 304 is woven through the base portion of main body 302 to act as a drawstring. In the example shown, a stopper 308 is positioned on each end portion of ligature 304 and is configured to be slid along the ligature 304 when an anchoring mechanism is temporarily released, such as by depressing a spring-loaded trigger 310. Upon release of trigger 310, stopper 308 resumes a friction grip against ligature 304 that prevents it from sliding with respect to ligature 304. Alternatively, both ends of ligature 304 may be threaded through a single stopper 308 that operates similarly.

After positioning main body 302 over the tissue walls 2 to be managed, as shown in FIG. 20B, the ends of ligature 304 are pulled in the direction of the arrow shown, while sliding stoppers against the elastomeric material of the base portion of device 300, thereby sealing off the tissue walls enclosed by device 300, in this case, atrial appendage 2. At this time, any manipulation performed on appendage 2 may be facilitated by the extra layer surrounding the tissue walls as provided by device 300. For example, bleeding caused by any incision or puncture through a tissue wall 2 is minimized by the elastomeric membrane 302 which acts as at least a partial seal after removal of the instrument used to perform the incision or puncture. FIG. 20C is a partial sectional view illustrating the sealing action by device layer 302 as needle 312 is removed from the puncture site after puncturing device wall 302 and tissue wall 2.

Additionally, surgical approaches have been developed in which one or more devices are inserted through an atrial appendage 2 to access the attached atrium 1 for a surgical procedure thereon. An example of such a procedure can be found in U.S. application Ser. No. 11/137,987 filed May 26, 2005, and titled “Ablation Instruments and Methods for Performing Ablation”. application Ser. No. 11/137,987 is hereby incorporated herein, in its entirety, by reference thereto. Not only does the elastomeric wall of device 300 function to manage such a procedure, including reduction or elimination of bleeding as described, but ligature 304 may be further cinched to contact tissue walls 2 against a tool having been inserted, to further manage the procedure, including reduction or elimination of bleeding past the contact between tissue walls 2 and the instrument.

Device 300 may also be employed to ligate tissue walls to prevent blood flow therepast. For example, FIG. 20D shows ligation of an atrial appendix 2 by further cinching ligature 304, relative to the position shown in FIG. 20B, so as to contact the tissue walls 2 together, thereby preventing blood flow therebetween. Stoppers 308 are anchored against ligature 304 and main body 302 in the positions shown to maintain the ligation at the base of the appendage 2. The ligation may be considered complete at this stage. Alternatively, an appendectomy may be performed as illustrated in FIG. 20E, using a surgical cutting instrument to remove the appendage 2 by cutting at a location 6 slightly above the site of the ligation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A device for occluding fluid flow between two walls of tissue in a patient, said device comprising:
a base configured to apply compressive force against a first of the two walls; and
at least one opposing member configured to apply compressive force against a second of the two walls upon installing the device, wherein, when installed, said base and said at least one opposing member compress the two walls therebetween, and a gap of predetermined dimension is established between said base and said at least one opposing member to sufficiently compress the two walls to occlude fluid flow therebetween, while preventing an establishment of a compression force that meets or exceeds a compression force to cause tissue necrosis.

2. The device of claim 1, wherein the two wall of tissue are opposing walls at the base of an atrial appendage.

3. The device of claim 1, comprising a clip, wherein said base comprises a first jaw of the clip and said at least one opposing member comprises a second jaw of the clip, said first and second jaws being joined at first ends thereof by a hinge, and second ends of said first and second jaws comprising a locking mechanism, wherein said first and second jaws are pivotable away from one another to an open position, and are pivotable towards one another to a closed position wherein said second ends are locked together via said locking mechanism, and wherein said gap of predetermined dimension is established in said closed position.

4. The device of claim 3, wherein said first and second jaws each have a length sufficient to exceed the length of a base portion of a left atrial appendage of a human heart.

5. The device of claim 3, further comprising compressible material lining inside surfaces of said first and second jaws, wherein said inside surface of said first jaw faces and opposes said inside surface of said second jaw.

6. The device of claim 3, further comprising a tool interface formed on each of said first and second jaws.

7. The device of claim 6, wherein said tool interfaces comprise loops extending from said first and second jaws.

8. The device of claim 3, wherein said first and second jaws extend longitudinally along a longitudinal axis, and wherein at least one pair of sides of said first and second jaws are curved relative to said longitudinal axis.

9. The device of claim 3, wherein a second of said pairs of sides of said first and second jaws are substantially straight and parallel to said longitudinal axis.

10. The device of claim 1, comprising a clip, wherein said base comprises a first jaw of the clip and said at least one opposing member comprises a second jaw of the clip, said first and second jaws being joinable at first ends thereof comprising a first locking mechanism, and second ends of said first and second jaws comprising a second locking mechanism, wherein said first and second jaws are movable away from one another in an open position, and are connectable to one another to assume a closed position wherein said first ends are locked together via said first locking mechanism and said second ends are locked together via said second locking mechanism, and wherein said gap of predetermined dimension is established in said closed position.

11. The device of claim 3, wherein at least one of said jaws includes a rib extending longitudinally therealong to reinforce said jaw against bending.

12. The device of claim 3, wherein at least one of said jaws is dished to provide additional rigidity, wherein edges of said jaw function as longitudinal strengthening ribs.

13. The device of claim 1, wherein at least one of said base and said at least one opposing member has at least one opening therein, wherein upon compressing said base and said at least one opposing member against the tissue walls, tissue protrudes through said at least one opening.

14. The device of claim 1, comprising a malleable clip, wherein said at least one opposing member comprises at least one tine extending from said base and having a distal tissue piercing end.

15. The device of claim 14, further comprising a pair of soft pads adapted to distribute compressive loads, wherein a first of said pair is positioned between said base and the first of the two walls, and a second of said pair is positioned between said at least one opposing member and the second of the two walls.

16. The device of claim 14, wherein said at least one tine extends from said base at an acute angle, wherein said device is configured to be slid laterally with respect to one of the tissue walls whereupon said at least one tine pierces the wall and is deformed toward a perpendicular orientation with respect to said base.

17. The device of claim 1, wherein said base comprises a first plate with a plurality of barbed connecting members extending therefrom, and said at least one opposing member comprises a second plate connectable to said first plate by insertion of said barbed connecting members therethrough, and wherein said gap of predetermined dimension is defined by a distance from said first plate to a proximal end of one of said barbs and a thickness of said second plate.

18. The device of claim 1, wherein said base comprises a first plate having first and second end portions with first and second connectors extending from said first and second end portions, and said at least one opposing member comprises a second plate connectable to said first plate by connection of said first and second connectors to first and second end portions of said second plate, and wherein said gap of predetermined dimension is defined by a length of said connectors and a thickness of said second plate.

19. The device of claim 1, further comprising an insertable body configured to be inserted between the two walls, wherein said base and said at least one opposing member each comprise connectors configured to connect to said insertable body and compress the first and second tissue walls against said insertable body, respectively.

20. The device of claim 17, further comprising a living hinge interconnecting said first and second plates.

21. The device of claim 17, wherein at least one of said barbed interconnecting members comprises a plurality of barbs, and wherein said gap of predetermined dimension is selectable from a plurality of predetermined dimensions dependent upon locations of said plurality of barbs.

22. The device of claim 1, wherein said base comprises a first clamping arm and said at least one opposing member comprises a second clamping arm, wherein said first and second clamping arms are interconnected by torsion arms.

23. The device of claim 1, wherein said base comprises a first clamping arm and said at least one opposing member comprises a second clamping arm, wherein said first and second clamping arms are interconnected by a torsion bar that is twistable about an axis of rotation when moving said first and second clamping arms apart from one another, said first and second clamping arms being rigid about said axis of rotation.

24. The device of claim 1, wherein a flexible stem extends from said base, said base being configured to be rotated to align with said flexible stem for insertion into a tubular delivery device.

25. The device of claim 1, wherein said base comprises a first plate and said at least one opposing member comprises a second plate cooperable with said first plate to compress the first and second walls together, said first and second plated being operable by flexible lines connected to one of said first and second plates and threaded through the other of said first and second plates, wherein flexible lines may be drawn upon to drive said first and second plates toward one another.

26. The device of claim 25, wherein at least one of said first and second plates comprises side walls extending therefrom, said side wall defining said predetermined dimension.

27. The device of claim 1, wherein said at least one opposing member comprises malleable tines extending from said base member, said device further comprising a tensioning member connected to said malleable tines and slidably passing through said base, wherein when tension is applied to said tensioning member and said base is held stationary, said malleable tines are deformed to compress toward said base.

28. The device of claim 1, wherein said base and said at least one opposing member comprise a pair of rigid, opposing elongated members, said elongated members being connected at one end by an elastic joint.

29. The device of claim 28, wherein said elongated members have an undulating shape to enhance friction against the tissue walls.

30. The device of claim 28, further comprising tabs extending from sides of said elongated members and adapted to be received in channels of a delivery tool.

31. The device of claim 1, wherein said base and said at least one opposing member comprise a pair of rigid, opposing elongated members, said elongated members being connected at one end by a malleable joint, said malleable joint being deformable to compress said elongated members against the tissue walls.

32. The device of claim 31, wherein said malleable joint is formed to have an acute angle bend and an obtuse angle bend.

33. The device of claim 1, wherein said base and said at least one opposing member comprise a pair of opposing elongated members, said elongated members being connected at one end by an elastically deformable joint and having free ends opposite of ends connected by said elastically deformable joint, said device further comprising a flexible line fixed to said free end of one of said elongated members and threadable through an opening in said free end of the other of said elongated members.

34. The device of claim 33, wherein said elongated members and said joint are formed of a shape memory material.

35. The device of claim 33, further comprising tool engagement features formed in said elongated members, said tool engagement features adapted to be engaged by a spreading tool.

36. The device of claim 35, wherein said tool engagement features comprise slots.

37. A device for occluding fluid flow between two walls of tissue in a patient, said device comprising:
a springform clip comprising at least three arms of substantially equal length, formed by bending an elongated member of spring metal, said arms being elastically deformable to insert the tissue walls therebetween, and having sufficient length to traverse an entire width of the tissue walls defining a passageway through which fluid flow occurs, wherein, upon releasing said arms from a deformed, open configuration, said arms elastically spring back toward an undeformed configuration to compress the tissue walls with sufficient force to occlude fluid flow therebetween, while preventing an establishment of a compression force that meets or exceeds a compression force to cause tissue necrosis.

38. The device of claim 37, further comprising traction features on at least one of said arms, to enhance friction between said device and the tissue walls compressed thereby.

39. A device for occluding fluid flow between two walls of tissue in a patient and a tool for installing said device, comprising:
a base configured to apply compressive force against a first of the two walls;
at least one opposing member configured to apply compressive force against a second of the two walls upon installing the device, wherein, when installed, said base and said at least one opposing member compress the two walls therebetween, and a gap of predetermined dimension is established between said base and said at least one opposing member to sufficiently compress the two walls to occlude fluid flow therebetween, while preventing an establishment of a compression force that meets or exceeds a compression force to cause tissue necrosis; and
a tool configured to interface with at least one of said base and at least one opposing member, to drive said device in compression against the tissue walls.

40. The device and tool of claim 39, further comprising a tool interface formed on said base and at least one said opposing member, wherein said tool comprises first and second arms moveable toward and away from one another, said first and second arms having control features configured to engage said tool interfaces form movement of said base and at least one said opposing member toward and away from one another.

41. The device and tool of claim 40, wherein said tool interfaces comprise loops, and said control features comprise tips of said arms.

42. The device and tool of claim 41, wherein said device comprises a clip, wherein said base comprises a first jaw of the clip and said at least one opposing member comprises a second jaw of the clip, said first and second jaws being joined at first ends thereof by a hinge, and second ends of said first and second jaws comprising a locking mechanism, wherein said first and second jaws are pivotable away from one another to an open position, and are pivotable towards one another to a closed position wherein said second ends are locked together via said locking mechanism, and wherein a gap of predetermined dimension is established in said closed position.

43. The device and tool of claim 42, wherein said first and second jaws each have a length sufficient to exceed the length of a base portion of a left atrial appendage of a human heart.

44. The device and tool of claim 41, wherein said tips are angled with respect to a remainder of said arms from which they extend.

45. The device and tool of claim 39, wherein said at least one opposing member comprises at least one malleable tine, and wherein said tool includes a pair of opposable jaws that are movable toward and away from one another, said tool further including mounting means on one of said jaws for mounting said base on said jaw, and wherein the other of said pair of jaws comprises an anvil against which said at least one malleable tine is deformed upon actuation of said tool to move said jaws toward one another to drive said at least one tine against said anvil.

46. The device and tool of claim 45, wherein said tool further comprises a stop configured to limit movement of said jaws towards one another to positions wherein a gap of predefined dimension is established between said jaws.

47. The device and tool of claim 39, wherein said device further comprises a flexible stem extending from said base, and said tool includes a tube, said stem being bendable for rotation of said base to orient said base to be inserted into said tube, and wherein said flexible stem has sufficient length to extend proximally from a proximal end of said tube when said base is outside of said tube and adjacent to said distal end of said tube, while said flexible member extends through said tube.

48. The device and tool of claim 47, wherein said at least one opposing member comprises a collar, wherein said flexible stem is threaded through said collar.

49. The device and tool of claim 47, comprising a slot in at least a distal portion of said tube.

50. The device and tool of claim 47, further comprising an elongated, rigid driver configured to pass through said tube and contact said base, wherein application of force through said driver to said base facilitates ejection of said base from said tube.

51. The device and tool of claim 48, further comprising a driver proximal of said collar and through which said flexible stem is threaded, said driver being slidable distally along said threaded member to move said collar toward said base.

52. The device and tool of claim 48, further comprising ratchet features on a distal portion of said flexible stem, said ratchet features configured to prevent sliding of said collar proximally along said flexible stem.

53. The device and tool of claim 39, wherein said device comprises a malleable clip, wherein said at least one opposing member comprises at least one tine extending from said base and having a distal tissue piercing end, and wherein said tool comprises a tool base against which said base of said device is positioned during installation of said device, and an anvil member having an elongated member slidably extending through said tool base and device, and an enlarged member, wherein, upon drawing said elongated member through said tool base, said enlarged member is driven against said at least one tine to deform said at least one tine into compression against one of the tissue walls.

54. The device and tool of claim 53, wherein said enlarged member of said anvil member comprises a sharpened distal end to facilitate piercing the tissue walls.

55. The device and tool of claim 39, wherein said base and said at least one opposing member comprise a pair of rigid, opposing elongated members, said elongated members being connected at one end by an elastic joint, and wherein said tool comprises opposing jaws mounted for articulation with respect to one another wherein said jaws are drivable together to compress the tissue walls therebetween in preparation for a ligation, said tool comprising a channel passing therethrough, said channel being dimensioned to slide said device therethrough for installation of said device over the compressed tissue walls.

56. The device and tool of claim 55, further comprising tabs extending from sides of said elongated members, and wherein said tool further comprises secondary channels in which said tabs are slidable, wherein said secondary channels guide said elongated members apart, into an open configuration as said device is being slid over the tissue walls, and then into a closed, clamping configuration at a final position for installation of said device.

57. The device and tool of claim 39, wherein said base and said at least one opposing member comprise a pair of rigid, opposing elongated members, said elongated members being connected at one end by a malleable joint, said malleable joint being deformable to compress said elongated members against the tissue walls, and wherein said tool comprises opposing jaws mounted for articulation with respect to one another wherein said jaws are drivable together to compress the tissue walls therebetween in preparation for a ligation, said tool comprising a channel passing therethrough, said channel being dimensioned to slide said device therethrough for installation of said device over the compressed tissue walls.

58. The device and tool of claim 57, further comprising an elongated pusher adapted to drive said device through said channel and to deform said malleable joint to compress said elongated members against the tissue walls.

59. The device and tool of claim 58, wherein said malleable joint, prior to deformation, comprises an acute angle bend and an obtuse angle bend, and wherein, compression of said elongated members by said pusher deforms said acute bend to form a smaller acute angle and increases the obtuse angle of the obtuse bend.

60. The device and tool of claim 39, further comprising a suction applicator, comprising an elongated tool body configured to be passed through a small thoracotomy, said suction applicator further comprising a tissue contacting member at a distal end thereof, said tissue contacting member being connectable to a suction source via a suction line extending the length of said suction applicator.

61. An assembly for performing an occlusion of fluid flow between two walls of tissue in a patient, said assembly comprising:
a device configured to maintain the two walls under sufficient compression to prevent fluid flow therebetween; and
a tool configured to guide installation of said device into a final configuration where said device maintains the two walls under said sufficient compression.

62. The assembly of claim 61, wherein said device comprises an elongated spiral shaped device, wherein and inside diameter of coils of said spiral shaped device is dimensioned to maintain the two walls under said sufficient compression, and wherein said tool comprises opposing jaws mounted for articulation with respect to one another wherein said jaws are drivable together to compress the tissue walls therebetween in preparation for a ligation, said tool comprising a channel passing therethrough, and pockets or threads to guide threading of said device therealong as said device is threaded through the compressed walls.

63. The assembly of claim 62, further comprising a rigid elongated driver dimensioned to be slidably received in said channel, said driver comprising a slot along a length thereof, a proximal end of said device extending inwardly of said inner diameter to engage with said slot.

64. The assembly of claim 61, wherein said device comprises a substantially straight, elongated needle having a sharpened distal end, and wherein said tool comprises opposing jaws mounted for articulation with respect to one another wherein said jaws have undulating opposing surfaces and are drivable together to compress the tissue walls therebetween in an undulating configuration, under said sufficient compression, said sufficient compression being less than an amount of compression to cause necrosis of the walls, in preparation for a ligation, said tool comprising a channel passing therethrough, wherein, upon passing said device through said channel with said jaws compressing the tissue walls, said device skewers the undulations of the tissue walls.

65. An assembly for performing an occlusion of fluid flow between two walls of tissue in a patient, said assembly comprising:
a multi-lumen endoscopic tool including an endoscope positionable in a lumen of said multi-lumen tool; a steerable suction tool positionable in another lumen of said multi-lumen tool such that a distal contact surface extends distally of a distal end of the multi-lumen tool; and a snare device insertable through another lumen of said multi-lumen tool, such that a snare at a distal end of said snare tool extends distally of said distal end of the multi-lumen tool.

66. A tissue wall coating device for minimizing bleeding caused by an incision or puncture of the tissue wall, said device comprising:
a main body in the shape of an elastomeric sack being closed at one end and having an opening at a base portion end; and
a ligature extending around said base portion end and configured to constrict said opening at said base portion end.

67. A method of performing an occlusion of fluid flow between two walls of tissue in a patient, said method comprising:
compressing the two walls together under compression force sufficient to prevent fluid flow between the two walls, but not so great to cause tissue necrosis; and
maintaining said compression force by installation of a device configured to maintain the walls between at least two contact surfaces separated from one another by a gap of predetermined dimension.

68. The method of claim 67, wherein said occlusion is performed on two walls of an atrial appendage.

69. The method of claim 67, wherein said occlusion is performed in a reduced-access surgical site.

70. The method of claim 67, wherein said device comprises a clip having opposing jaws, and wherein said compressing and maintaining said compression force comprises extending said clip over an entire width of the tissue walls, moving said opposing jaws toward one another to establish said compression force and locking relative positions of said jaws in compression against the two walls, wherein a gap of predetermined dimension is established and maintained between said jaws.

71. The method of claim 70, further comprising releasing said locking of the relative positions, repositioning said jaws relative to the tissue walls and locking relative positions of said jaws again to establish and maintain said gap.

72. The method of claim 67, further comprising laterally sliding, relative to one of the tissue walls, a portion of the device comprising a plate having tines extending therefrom at an acute angle, wherein said tines pierce the tissue wall;
continuing said lateral sliding to increase the angle of said tines with respect to said plate toward perpendicular;
forcing said tines and said plate toward the tissue wall to pierce both walls of tissue with said tines and cause distal end of said tines to extend from an opposite surface of the second wall of tissue;
wherein said compressing comprises deforming said tines against the opposite surface to compress the two walls between the deformed tines and said plate.

73. The method of claim 72, further comprising placing a first pad between over said tines and against said plate prior to said lateral sliding, and placing a second pad over said tines and against the opposite surface prior to said deforming said tines, wherein the tissue walls are compressed between said first and second pads.

74. The method of claim 67, wherein said device comprises opposing contact surfaces connected by connecting members having ratcheting features, said method further comprising varying a distance between said opposing contact surfaces in a location of at least one of said connecting members, wherein at least two different gaps of different predetermined dimensions are maintained at different locations along said contact surfaces.

75. The method of claim 67, wherein the tissue walls are the tissue walls along the base of an atrial appendage, said method further comprising:
folding over the atrial appendage after occluding flow between the tissue walls at the base; and
attaching the folded over tissue of the atrial appendage to the adjacent atrium or other adjacent tissue.

76. The method of claim 67, wherein the tissue walls are the tissue walls along the base of an atrial appendage, said method further comprising:
twisting and folding over the atrial appendage after occluding flow between the tissue walls at the base; and
attaching the twisted, folded over tissue of the atrial appendage to the adjacent atrium or other adjacent tissue.

77. The method of claim 67, further comprising piercing through the two walls of tissue with a tubular member having a sharpened distal end;
ejecting a base member connected to a flexible stem from said sharpened distal end, whereby said flexible stem extends through the two walls of tissue;
removing said tubular member from the tissue walls; and
wherein said compressing and maintaining are carried out by sliding a collar over said flexible stem and against a first of the tissue walls wherein the tissue walls are compressed between said collar and base, and fixing a position of said collar relative to said base to define said gap.

78. The method of claim 67, wherein said device comprises a pair of plates, further comprising providing an opening between said pair of plates sufficient to allow said plates to be positioned over the two tissue walls;
orienting said plates adjacent to locations on the tissue walls where the occlusion is desired;
wherein said compressing is carried out by drawing on connecting members attached to one of said plates and threaded through the other, to draw said plates together in compression; and
wherein said maintaining is effected by fixing said connecting members relative to said plate that said connecting members are threaded through.

79. The method of claim 67, wherein said compressing comprises clamping opposing jaws of a delivery tool over the two walls, wherein the delivery tool is configured to prevent complete closure of the opposing jaws together, being stopped to define a predetermined gap therebetween; and wherein said maintaining is effected by sliding a device through a channel formed in said delivery tool, over the two tissue walls, wherein said device maintains the tissue walls between opposing members separated by said gap of predetermined dimension.

80. The method of claim 79, further comprising removing said delivery tool from the two walls of tissue.

81. The method of claim 79, further comprising crimping said device to compress the opposing members toward one another to assume locations defining said gap of predetermined dimension.

82. The method of claim 67, further comprising contacting at least one of the tissue walls with a contact member of a manipulator, at a location in the vicinity where the occlusion is to be performed, applying suction through the contact member to attaché the contact member to the tissue wall; and moving the tissue by moving the manipulator, to facilitate said compressing and maintaining steps.

83. The method of claim 67, wherein said device comprises a clip having opposing jaws, and wherein said compressing comprises extending said clip over an entire width of the tissue walls with said opposing jaws being held in an open, elastically deformed configuration, and releasing said opposing jaws to permit said jaws to moving toward one another to establish said compression force; and
wherein said maintaining said compression force includes drawing a tie line against the tissue walls, between open ends of said opposing jaws.

84. The method of claim 67, wherein said compressing comprises clamping opposing jaws of a delivery tool over the two walls, wherein the delivery tool is configured to prevent complete closure of the opposing jaws together, being stopped to define a predetermined gap therebetween; and wherein said maintaining is effected by screwing a helical device through the two walls, wherein said screwing is guided by pockets or threads formed in the opposing surfaces of said jaws.

85. The method of claim 84, further comprising removing said delivery tool from the two walls of tissue.

86. The method of claim 67, wherein said compressing comprises clamping opposing jaws of a delivery tool over the two walls, wherein the delivery tool is configured to prevent complete closure of the opposing jaws together, being stopped to define a predetermined gap therebetween; and wherein said maintaining is effected by screwing a helical device through the two walls, wherein said screwing is guided by pockets or threads formed in the opposing surfaces of said jaws, and wherein said helical device has a removable base mounted on a distal end thereof, said removable base further comprising a flexible member attached thereto and extending proximally thereof, such that upon screwing said base through the two tissue walls, the flexible member extends through all pierced turns of the screw installation and proximally of an entry point in the tissue walls where the screwing was initiated,
withdrawing said helical member from the tissue walls, while maintaining the removable base distally of the distal most piercing; and
knotting or otherwise anchoring said flexible member in a location adjacent the entry point.

87. The method of claim 67, wherein said compressing comprises the two walls of tissue into an undulating conformation using opposing jaws, having undulating opposing surfaces, of a delivery tool, wherein the delivery tool is configured to prevent complete closure of the opposing jaws together, being stopped to define a predetermined gap therebetween; and wherein said maintaining is effected by skewering the undulations of compressed tissue walls with a substantially straight needle.

88. The method of claim 67, wherein said compressing comprises the two walls of tissue into an undulating conformation using opposing jaws, having undulating opposing surfaces, of a delivery tool, wherein the delivery tool is configured to prevent complete closure of the opposing jaws together, being stopped to define a predetermined gap therebetween; and wherein said maintaining is effected by skewering the undulations of compressed tissue walls with a substantially straight needle, having a removable base mounted on a distal end thereof, said removable base further comprising a flexible member attached thereto and extending proximally thereof, such that upon skewering the undulations, the flexible member extends through all pierced undulations and proximally of an entry point in the tissue walls where the skewering was initiated,
withdrawing said needle from the undulations, while maintaining the removable base distally of the distal most piercing; and
knotting or otherwise anchoring said flexible member in a location adjacent the entry point.

89. A method of performing an occlusion of fluid flow between two walls of tissue in a patient through a small opening in the patient, said method comprising:
inserting a multi-lumen endoscopic tool through the small opening in the patient, toward a location of the two walls of tissue to be occluded;
identifying the location of the two walls of tissue to be occluded by viewing through an endoscope inserted in one of the lumens of the multi-lumen tool, and maneuvering a distal end of the multi-lumen tool to a reduced-access sited containing the two walls of tissue;
snaring the tissue walls with a snare extending at the distal end of a snare tool inserted through another lumen of the multi-lumen device;
tightening the snare from a location outside of the patient;
fixing the snare in the tightened configuration to maintain compression of the two walls; and
cutting the fixed snare away from the snare device.

90. The method of claim 89, wherein the two walls are at the base of an atrial appendage.

91. The method of claim 89, further comprising applying suction, via a suction manipulator to attach to a wall of the tissue and manipulate the tissue to facilitate at lest one of said snaring, tightening, fixing and cutting.

92. A method of reducing bleeding during a surgical procedure on an atrial appendix, said method comprising:
placing an elastomeric sack over the atrial appendix to form a slightly compressive interface between said sack and the atrial appendix;
constricting a base opening of the sack by drawing on a ligature passing through the base opening, and thereby compressing the walls of tissue at the location contacted by the base opening; and
fixing said ligature relative to the base opening to maintain said constricting.

93. The method of claim 92, further comprising excising a portion of the sack and the atrial appendage at a location above said base opening.
